(19) United States
(12) Patent Application Publication
(10) Pub. No.: US 2025/0273291 A1
(43) Pub. Date: Aug. 28, 2025

(54) SELECTION OF DIVERSE CANDIDATE PEPTIDES FOR PEPTIDE THERAPEUTICS

(71) Applicant: Genentech, Inc., South San Francisco, CA (US)

(72) Inventors: William John THRIFT, San Francisco, CA (US); Kai LIU, Belmont, CA (US)

(21) Appl. No.: 19/207,999

(22) Filed: May 14, 2025

Related U.S. Application Data
(63) Continuation of application No. PCT/US2023/079683, filed on Nov. 14, 2023.
(60) Provisional application No. 63/425,647, filed on Nov. 15, 2022.

(51) Int. Cl. G16B 15/30; G06N 20/00; G16B 40/20; G16H 15/00; G16H 20/17

(52) U.S. Cl. CPC G16B 15/30; G06N 20/00; G16B 40/20; G16H 15/00; G16H 20/17

(57) ABSTRACT

A method for developing a therapeutic such as, for example, a peptide vaccine. A machine learning model is trained using a metric learning algorithm, training peptide sequence data, and training allele presentation data corresponding to the training peptide sequence data. Peptide sequence data identifying peptide sequences that correspond to peptides is received. A peptide sequence vector is generated, via a machine learning model, for each peptide sequence using the peptide sequence data to form a plurality of peptide sequence vectors. An output is generated using the plurality of peptide sequence vectors. The output provides an indication of similarity between peptide sequences of the plurality of peptide sequences. A group of candidate peptides is selected from the plurality of peptides for development of the therapeutic based on the output such that the group of candidate peptides includes at least two dissimilar candidate peptides.

### CROSS-REFERENCE TO RELATED APPLICATIONS

This application is a continuation of International Application No. PCT/US2023/079683, filed on Nov. 14, 2023, which claims priority to U.S. Provisional Patent Application No. 63/425,647, entitled “Selection of Diverse Candidate Peptides for Peptide Therapeutics,” filed Nov. 15, 2022, the disclosure of which is incorporated herein by reference in its entirety.

### TECHNICAL FIELD

The present disclosure generally relates to selecting candidate peptides for developing therapeutics. More particularly, the present disclosure relates to machine learning-based methods and systems for selecting a group of candidate peptides that is diverse with respect to binding motifs for use in developing peptide immunotherapies (e.g., peptide therapeutics such as peptide vaccines, cell therapies, etc.) with improved efficacy.

### BACKGROUND

Personalized immunotherapies include treatments that use an individual's own immune system to fight diseases such as cancer. Immunotherapies may include, for example, peptide therapeutics (e.g., peptide vaccines) and cell therapies. Peptide vaccines, for example, are made using one or more peptides that mimic the epitopes of antigens that trigger immune responses. Peptide vaccines can be used to induce protection against infectious pathogens and non-infectious diseases and can be used as therapeutic cancer vaccines (e.g., neoantigen vaccines). Neoantigen vaccines are a relatively new approach for providing individualized cancer treatment in which peptides from tumor-associated antigens are used to induce an effective anti-tumor T-cell response. Cell therapies may involve injecting cells (e.g., T cells or tumor cells) into an individual to generate or elicit an immune response. For example, T cells may be collected from an individual's blood and modified to produce a more vigorous attack on the tumor cells. These T cells may then be injected into the individual to generate the desired immune response. As another example, tumor cells may be collected from the individual and re-engineered to draw out an attack by the immune system.

Selecting candidate peptides for use in developing personalized immunotherapies can be challenging. Currently available methods for identifying and ranking candidate peptides for use in the development of a personalized immunotherapy may be difficult in that numerous peptide sequences may be detected in a given sample. Knowing whether a given peptide is similar to another peptide may be useful in the identification and ranking of candidate peptides. Thus, it may be desirable to have methods and/or systems for evaluating the similarity between peptides to aid in the selection of candidate peptides to be used in the development of personalized immunotherapies with improved efficacy.

### SUMMARY

In one or more embodiments, a method is provided for developing a therapeutic. Peptide sequence data identifying a plurality of peptide sequences that correspond to a plurality of peptides is received. A plurality of peptide sequence vectors are generated, via a machine learning model, in an n-dimensional space for respective ones of the peptide sequences and thereby, for respective ones of the peptides. The machine learning model has been trained using a metric learning algorithm, training peptide sequence data, and training allele presentation data. The training peptide sequence data identifies a training peptide sequence that corresponds to each training peptide of a plurality of training peptides. The training allele presentation data identifies, for each training peptide sequence in the training peptide sequence data, one or more major histocompatibility complex (MHC) alleles expected to present the training peptide that corresponds to the training peptide sequence. The metric learning algorithm has been used to train the machine learning model such that a first distance between a first pair of the peptide sequence vectors generated within the n-dimensional space for a first respective pair of the peptides that are presented by a same MHC allele is less than a second distance between a second pair of the peptide sequence vectors generated within the n-dimensional space for a second pair of the peptides that are presented by different MHC alleles. An output is generated using the peptide sequence vectors. The output provides an indication of similarity between the peptide sequences for use in selecting a diverse group of candidate peptides from the peptides for development of the therapeutic.

In one or more embodiments, a method is provided for developing a peptide vaccine. A machine learning model is trained using a metric learning algorithm, training peptide sequence data, and training allele presentation data corresponding to the training peptide sequence data. Peptide sequence data identifying a plurality of peptide sequences that correspond to a plurality of peptides is received. A peptide sequence vector is generated for each peptide sequence of the plurality of peptide sequences, via a machine learning model, using the peptide sequence data to form a plurality of peptide sequence vectors. An output is generated using the plurality of peptide sequence vectors. The output provides an indication of similarity between peptide sequences of the plurality of peptide sequences. A diverse group of candidate peptides is selected from the plurality of peptides for development of the peptide vaccine based on the output such that the diverse group of candidate peptides includes at least two dissimilar candidate peptides.

In one or more embodiments, a method is provided that includes: receiving training peptide sequence data comprising a plurality of training peptide sequences; generating training allele presentation data for the training peptide sequence data, wherein the training allele presentation data identifies, for a training peptide sequence of the plurality of training peptide sequences, an MHC allele that is predicted to present the training peptide sequence; and training a machine learning model using the training peptide sequence data, the training allele presentation data, and a metric learning algorithm. The machine learning model is trained to generate a peptide sequence vector for a given peptide sequence. The peptide sequence vector is a vector in an n-dimensional space that provides an indication of a similarity of the given peptide sequence to other peptide sequences.

In one or more embodiments, a vaccine comprises a plurality of peptides, a plurality of precursors for the plurality of peptides, or a set of nucleic acids encoding the plurality of peptides or the plurality of precursors. The plurality of peptides includes at least two peptides that have dissimilar binding motifs. The plurality of peptides are selected from among a diverse group of candidate peptides that is selected based on part of or all of one or more of the methods described herein.

In one or more embodiments, a method of manufacturing a vaccine is provided. The vaccine comprises a plurality of peptides, a plurality of precursors for the plurality of peptides, or a set of nucleic acids encoding the plurality of peptides or the plurality of precursors. The plurality of peptides includes at least two peptides that have dissimilar binding motifs. The plurality of peptides are selected from among a diverse group of candidate peptides that is selected based on part of or all of one or more of the methods described herein.

In one or more embodiments, a pharmaceutical composition that includes two or more peptides selected from a diverse group of candidate peptides that is selected based on part of or all of one or more of the methods described herein.

In one or more embodiments, a pharmaceutical composition that includes two or more nucleic acid sequences that encodes two or more respective peptides selected from the diverse group of candidate peptides that is selected based on part of or all of one or more of the methods described herein.

In one or more embodiments, a method of treating a subject is provided. The method includes administering at least one of two or more peptides, two or more pharmaceutical compositions, or two or more nucleic acid sequences identified based on the output generated by part of or all of one or more of the methods described herein.

In one or more embodiments, an engineered T cell is made using part of or all of one or more of the methods described herein.

In one or more embodiments, a population of engineered T cells is made using part of or all of one or more of the methods described herein.

In one or more embodiments, a method is provided for treating a subject having cancer. A population of T cells is provided. At least a subset of the population of T cells is engineered to express an exogenous T cell receptor (TCR) and to knock out an endogenous TCR-beta, thereby forming a population of engineered T cells. The exogenous TCR binds to an antigen expressed by the cancer and selected using part of or all of one or more of the methods described herein. The population of engineered T cells is expanded. The expanded population of engineered T cells is administered to the subject.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1 is a block diagram of a therapeutic development system 100 in accordance with various embodiments.

FIG. 2 is a schematic diagram of one example of a configuration for the model in FIG. 1 in accordance with one or more embodiments.

FIG. 3 is a flowchart of a process for use in developing a therapeutic in accordance with one or more embodiments.

FIG. 4 is a flowchart of a process for training a machine learning model to generate peptide sequence vectors for peptide sequences in accordance with one or more embodiments.

FIG. 5 is a flowchart of a process for training a machine learning model to generate peptide sequence vectors for peptide sequences in accordance with one or more embodiments.

FIG. 6 is a flowchart of a process for treating a subject in accordance with one or more embodiments.

FIG. 7 is an illustration of a plot of peptide sequence vectors in a reduced dimensional space in accordance with one or more embodiments.

FIG. 8 is a listing of MHC alleles in accordance with one or more embodiments.

FIG. 9 is a block diagram of a computer system in accordance with various embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

### I. Overview

Recognizing the importance of being able to determine which peptides to select as candidates for use in the development of personalized immunotherapies (e.g., cell therapies, peptide therapeutics such as peptide vaccines, etc.), the embodiments described herein provide methodologies and systems for making such determinations in a manner that leads to improved therapeutic efficacy as compared to various currently available methods and systems. The embodiments described herein use machine learning methodologies and systems to improve peptide selection performance by, for example, without limitation, increasing the diversity of binding motifs of the candidate peptides selected for use in the development of a therapeutic. Increasing binding motif diversity may increase the likelihood of presentation of one or more of these peptides. The binding motif of a peptide may be a particular configuration (or “motif”) of amino acids that allows the peptide to be bound to and presented by a corresponding major histocompatibility complex (MHC) allele. In humans, these MHC alleles are referred to as human leukocyte antigen (HLA) alleles.

References herein to MHC alleles or HLA alleles refer to the proteins defined by these alleles. For example, an MHC allele that is described as presenting or capable of presenting a peptide may refer to the protein defined by the MHC allele that has a binding pocket (or binding groove) that can bind to a corresponding binding motif of that peptide. The binding pocket may be a particular sequence configuration that can bind to a corresponding binding motif. A peptide may have one or more binding motifs that allow the peptide to be bound to one or more respective MHC alleles. Further, two or more peptides may have a binding motif in common. Thus, an MHC allele that can present or is capable of presenting a peptide may also be described as presenting or capable of presenting a peptide sequence corresponding to that peptide. In other words, the protein defined by that MHC allele can bind to a peptide having that peptide sequence.

The embodiments described herein provide a machine learning model and various methodologies of using the machine learning model and/or the output generated by the machine learning model to analyze peptide sequences identified from one or more samples from one or more subjects. A peptide sequence is a sequence that corresponds to at least a portion of a peptide, the sequence being an amino acid sequence, a codon sequence, or a nucleic acid sequence. A sample may be, for example, but is not limited to, a disease sample (e.g., diseased tissue, tumor tissue). Peptide sequences detected or identified from these one or more samples may be processed using a machine learning model to generate mathematical representations of these peptide sequences that capture information about the peptide sequences and thereby, the peptides that have these peptide sequences. Further, because binding motifs are sequence-based, these mathematical representations also capture information about the one or more binding motifs contained within a given peptide sequence.

In particular, the machine learning model processes the peptide sequences to generate mathematical representations in the form of peptide sequence vectors, which may also be referred to as peptide sequence embeddings. A peptide sequence vector (or PS vector) generated for a peptide sequence may also be referred to as being for or corresponding to the peptide having the peptide sequence. A peptide sequence vector is a vector that has n dimensions (or n discrete elements) within an embedding space. In other words, these peptide sequence vectors are vectors within an n-dimensional (embedding) space.

The distance between any two peptide sequence vectors with respect to this n-dimensional space may provide some indication of how similar or dissimilar the corresponding peptides are relative to each other. Peptide sequence vectors that represent similar peptide sequences are embedded closer together within the n-dimensional space, while peptide sequence vectors that represent dissimilar peptide sequences are embedded further apart within the n-dimensional space. When two peptide sequences are similar, their corresponding peptides may also be referred to as being similar. Further, because binding motifs are sequence-based, two peptides that are similar to each other as determined by their peptide sequence vectors may be considered as having similar binding motifs. Two peptides that are dissimilar as determined by their peptide sequence vectors may be considered as having dissimilar binding motifs. In some cases, a peptide may have more than one binding motif.

The embodiments described herein recognize that metric learning may be used to improve the training and performance of the machine learning model in differentiating between the binding motifs of different peptide sequences. Accordingly, in one or more embodiments, the machine learning model is trained to generate the peptide sequence vectors for the peptide sequences using a metric learning algorithm. The metric learning algorithm uses one or more loss functions, which may include, for example, without limitation, at least one of a contrastive loss function, a triplet loss function, a quadruplet loss function, a circle loss function, a multi-class n-pair loss function, a lifted structure loss function, an angular loss function, a divergence loss function, a constellation loss function, or some other type of distance-based loss function.

Using information in the metric learning algorithm about the MHC alleles that are expected to (or predicted to) present peptides improves the performance of the training. Thus, the metric learning algorithm is used to train the machine learning model to generate peptide sequence vectors for peptides that are presented by a same MHC allele closer together in the n-dimensional space and to generate peptide sequence vectors for peptides that are presented by different MHC alleles further away from each other in the n-dimensional space.

Using the peptide sequence vectors generated by the machine learning model, which has been trained using the metric learning algorithm, an output can then be generated that provides an indication of the similarity between peptides. The output may directly present the peptide sequence vectors generated by the machine learning model or may present the peptide sequence vectors in a form that can be more readily understood or deciphered by a human. For example, the output may take the form of a visual representation, a spreadsheet, a list, or some other type of output.

In one or more embodiments, the output takes the form of a graphical representation of the peptide sequence vectors in a k-dimensional space. The k-dimensional space may include the same or fewer dimensions than the n-dimensional space of the peptide sequence vectors (i.e., k≤n). Presenting the peptide sequence vectors in a reduced dimensional space may allow a human to more quickly and/or easily understand or ascertain the similarity relationships between the corresponding peptides. For example, the n-dimensional space may include between 20 and 200 dimensions. In some embodiments, the output presents the peptide sequence vectors with respect to 2 or 3 dimensions for ease of visualization and understanding.

The output that is generated may be used to select a diverse group of candidate peptides for the development of a personalized immunotherapy. For example, a personalized immunotherapy (e.g., peptide vaccine, cell therapy, etc.) may be created using the entire diverse group of candidate peptides or may be created using at least two peptides of the diverse group of candidate peptides, the at least two peptides being dissimilar. The diversity in the group of candidate peptides may include, for example, but is not limited to, binding motif diversity. For example, at least two peptides in the diverse group of candidate peptides may have dissimilar binding motifs. The personalized immunotherapy may be created with at least two peptides that have dissimilar binding motifs.

A personalized immunotherapy (e.g., a peptide vaccine, cell therapy, etc.) that is developed using a diverse group of candidate peptides may have improved efficacy because this diversity may allow the immunotherapy to be applicable where biological circumstances have led to certain binding motifs being favored over what is expected. For example, a tumor or diseased tissue's microenvironment (e.g., pH, temperature, etc.) may lead to one or more different binding motifs being favored over what may have been seen via the processing of various samples via mass spectrometry. Further, individual subjects may have biological differences that lead to one or more different binding motifs being favored over what may have been seen via the processing of various samples via mass spectrometry. Developing a peptide vaccine using a group of candidate peptides with diverse binding motifs increases the chances of triggering of the desired immunological response. For example, having diverse peptides in a peptide vaccine may increase the chances of presentation via MHC alleles and thereby, increase the chances of triggering the desired immunological response and increase the strength of the immunological response that is triggered.

The embodiments described herein further recognize and take into account that training a model for sequence analysis may be particularly complicated due to the sheer number of peptide sequences potentially observable. Not only are there millions of potential presented peptides (e.g., neoantigens), but the genes encoding the proteins for MHC class I molecules, for example, are also highly polymorphic: there are nearly 20,000 alleles of human MHC class I. Thus, the embodiments described herein provide methodologies and systems for training the machine learning model in a manner that improves the overall performance and efficiency of selecting a group of candidate peptides for a peptide vaccine that will have improved chance of success. The machine learning model is trained to embed the peptide sequences for the peptides in the n-dimensional space such that the further apart two embeddings are, the more dissimilar they are. Candidate peptides are selected in a manner that maximizes the distance between the embeddings representing these candidate peptides or that ensures distances above a certain distance threshold to ensure diversity. Candidate peptides are selected to ensure diverse binding motifs but may also be selected to ensure clearly defined and/or well-known binding motifs.

### II. Example System for Diversifying Peptides Used in the Development of a Peptide Therapeutic (e.g., Peptide Vaccine)

### II.A. Example Therapeutic Development System

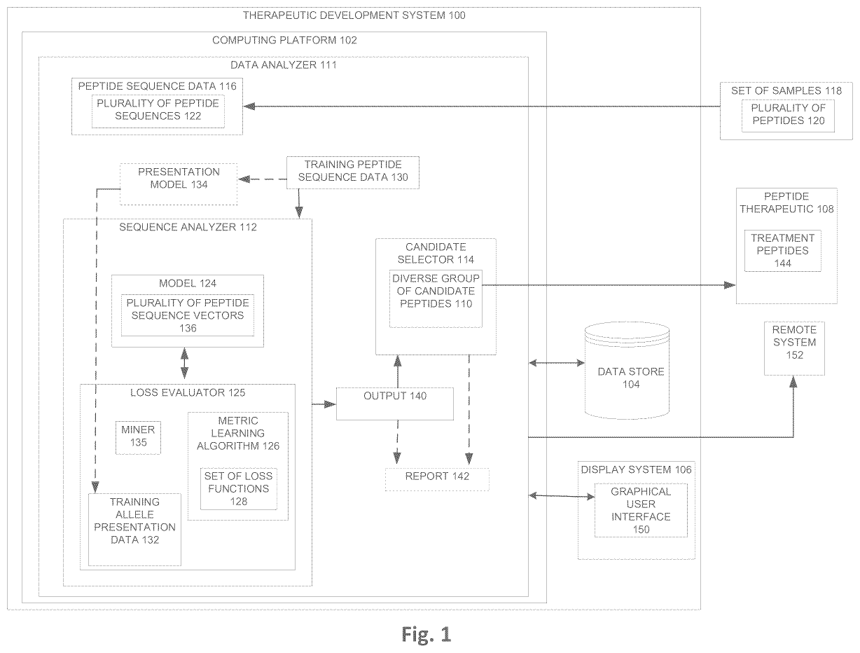

Referring now to the figures, FIG. 1 is a block diagram of a therapeutic development system 100 in accordance with various embodiments. Therapeutic development system 100 includes computing platform 102, data store 104, and display system 106. Computing platform 102 may take various forms. In one or more embodiments, computing platform 102 includes a single computer (or computer system) or multiple computers in communication with each other. In other examples, computing platform 102 takes the form of a cloud computing platform.

Data store 104 and display system 106 are each in communication with computing platform 102. In some examples, data store 104, display system 106, or both may be considered part of or otherwise integrated with computing platform 102. Thus, in some examples, computing platform 102, data store 104, and display system 106 may be separate components in communication with each other, but in other examples, some combination of these components may be integrated together. Communication between the different components may be implemented using any number of wired communications links, wireless communications links, optical communications links, or a combination thereof.

Therapeutic development system 100, which may also be referred to as a peptide therapeutic development system, is used to develop peptide therapeutic 108. Peptide therapeutic 108 may be, for example, a peptide vaccine that includes multiple peptides, precursors for the multiple peptides, or one or more nucleic acids that encode the multiple peptides or precursor thereof. In one or more embodiments, the peptide vaccine may be an individualized vaccine. The peptide vaccine may be, for example, a neoantigen vaccine that includes multiple neoantigens selected for treating cancer. The neoantigen vaccine may be engineered or selected based on a subject-specific tumor profile of peptides.

Therapeutic development system 100 is used to select a diverse group of candidate peptides 110 for use in creating peptide therapeutic 108. A diverse group of candidate peptides 110 includes at least two peptides that are dissimilar. In one or more embodiments, the at least two peptides may be dissimilar with respect to their binding motifs. For example, at least two peptides in diverse group of candidate peptides 110 may have dissimilar binding motifs. Peptide therapeutic 108 may be created using at least two peptides from diverse group of candidate peptides 110, these at least two peptides being dissimilar (e.g., have dissimilar binding motifs).

Binding motifs may be dissimilar when the amino acids (or sequence of amino acids) of the binding motif are different, the spacing (or intervals) between the amino acids is different, or a combination thereof. For example, the binding motifs may be dissimilar by differing by more than a selected number of amino acids (e.g., two, three, four, five, or more amino acids). Thus, the dissimilarity between the binding motifs of peptides may vary by degree. Two binding motifs that differ by a single amino acid are less dissimilar than two binding motifs that differ by three or four amino acids.

Therapeutic development system 100 includes data analyzer 111. Data analyzer 111 may be implemented using hardware, software, firmware, or a combination thereof. In one or more embodiments, data analyzer 111 is implemented in computing platform 102.

Data analyzer 111 may include, for example, without limitation, sequence analyzer 112 and candidate selector 114, each of which may be implemented using hardware, software, firmware, or a combination thereof. In some embodiments, sequence analyzer 112 and candidate selector 114 are integrated together within a same module in data analyzer 111. Sequence analyzer 112 is used to evaluate the similarity and/or dissimilarity of different peptides. Candidate selector 114 is used to select diverse group of candidate peptides 110 for peptide therapeutic 108 based on the analysis performed by sequence analyzer 112.

Data analyzer 111 may receive peptide sequence data 116 (e.g., over one or more wired, wireless, and/or optical communications links), retrieve peptide sequence data 116 from data store 104 or some other type of storage (e.g., cloud storage), access peptide sequence data 116 from multiple types of storage, generate peptide sequence data 116 based on the results of mass spectroscopy, and/or obtain peptide sequence data 116 in some other manner. In one or more embodiments, peptide sequence data 116 may be retrieved from data store 104 in response to receiving user input entered by a user via an input device.

In one or more embodiments, peptide sequence data 116 is generated from the processing of a set of samples 118. Set of samples 118 may take the form of one or more biological samples from one or more subjects (e.g., a disease (or diseased) sample, a healthy sample, a combination thereof). In one or more embodiments, set of samples 118 includes a sample obtained from a tumor of a subject. The tumor may be a manifestation of, for example, lung cancer, melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, T cell lymphocytic leukemia, non-small cell lung cancer, small-cell lung cancer, another type of cancer, or a combination thereof.

Set of samples 118 may be processed together to generate peptide sequence data 116. In some instances, multiple samples in set of samples 118 may be processed at different times to generate peptide sequence data 116. In some embodiments, therapeutic development system 100 includes a sample analyzer that is used in the processing of set of samples 118 to generate peptide sequence data 116. The sample analyzer may include, for example, without limitation, a mass spectrometry system.

Peptide sequence data 116 identifies plurality of peptide sequences 122 that are detected within set of samples 118. Each peptide sequence of the peptide sequences 122 that is detected characterizes at least a portion of a corresponding peptide. In other words, each peptide sequence forms at least a portion of a peptide. The peptide sequence may be, for example, without limitation, an amino acid sequence, a nucleic acid sequence, or a codon sequence. A peptide, such as a peptide of the plurality of peptides 120, may be a mutant peptide (e.g., neoantigen) when the peptide includes one or more variants (e.g., one or more sequence variations) compared to a corresponding reference sequence. In other words, the mutant peptide has a peptide sequence that includes a variant-coding sequence that includes at least one variant with respect to a corresponding reference sequence.

In one or more embodiments, sequence analyzer 112 of data analyzer 111 receives peptide sequence data 116 as input for processing. Sequence analyzer 112 includes model 124 that processes peptide sequence data 116 and loss evaluator 125 that is used to train model 124. In some embodiments, peptide sequence data 116 is sent directly into model 124 for processing. In other embodiments, sequence analyzer 112 preprocesses peptide sequence data 116 prior to sending peptide sequence data 116 into model 124 for processing.

Model 124 may be comprised of any number or combination of models, algorithms, functions, etc. In one or more embodiments, model 124 includes a machine learning model that can be implemented in any of a number of different ways. For example, model 124 may include a deep learning model. The deep learning model may include, for example, without limitation, one or more convolutional neural networks, one or more recurrent neural networks, one or more feed forward neural networks, one or more other types of neural networks, or a combination thereof.

In one or more embodiments, model 124 includes various subsystems of processing. Each “subsystem” may be comprised of one or more blocks, with each block being comprised of one or more sub-blocks and/or layers. A sub-block may be comprised of any number of layers (or units).

In one or more embodiments, model 124 includes an encoder-decoder model. For example, model 124 may include a sequence to sequence (seq2seq) learning model, which may be implemented in any of a number of different ways. For example, the sequence to sequence learning model may take the form of an attention-based machine learning model (e.g., includes one or more attention layers). The sequence to sequence learning model may include, but is not limited to, one or more recurrent neural networks. For example, the sequence to sequence learning model may include a multi-layered Long Short-Term Memory model.

Model 124 may include multiple subsystems (or subnetworks). Each of the multiple subsystems can include an encoder, a transformer, a transformer encoder, one or more attention layers, and/or one or more self-attention layers. For example, model 124 may include one or more encoders configured to, for example, transform an input (e.g., a sequence representation representing, for example, an amino acid sequence, a nucleic acid sequence, a codon sequence, etc.) into a higher dimensional space. An encoder may be a transformer encoder. The encoder may be configured to implement an attention-based technique and/or to include one or more attention layers (e.g., one or more self-attention layers). Model 124 can use a self-attention mechanism, a global attention mechanism, a soft attention mechanism, a local attention mechanism, and/or a hard attention mechanism. Model 124 may include one or more functions such as, for example, without limitation, at least one of a content-based function, an additive function, a location-based function, a dot-product function, a scaled dot-product function, or another function.

In one or more embodiments, model 124 is trained using metric learning to learn a representation function that maps an input peptide sequence to a peptide sequence vector in an embedded space. Accordingly, model 124 may be referred to as a metric learning model (e.g., a deep metric learning model). The embedded space may be an n-dimensional space. Thus, the peptide sequence vector may be a vector of n dimensions (or of n discrete elements). Such a vector may also be referred to as an embedding. The n-dimensional space may include, for example, without limitation, 2, 5, 10, 20, 30, 50, 75, 100, 128, 200, 256, 300, 400, 450, 500, 800, 1024, 1600, 2048, 2500, or some other number of dimensions (e.g., up to 3000 dimensions).

The distances between the peptide sequence vectors (or embeddings) for various peptide sequences in the embedded space preserve the similarity of the peptide sequences (and thereby, the corresponding peptides). For example, the distance (a computed distance metric) between any two peptide sequence vectors in the embedded space provides an indication of how similar the corresponding peptide sequences (and thereby corresponding peptides) are relative to each other. This distance may be, for example, but is not limited to, Euclidean distance, a cosine distance, or some other type of distance metric. A shorter distance between two peptide sequence vectors indicates that the corresponding peptides are more similar, whereas a greater distance means the corresponding peptides are less similar (or more dissimilar).

For example, the position of a peptide sequence vector within the embedded space may provide information about the binding characteristics and/or capabilities of the corresponding peptide. For example, two peptide sequence vectors that are close together in the embedded space may correspond to peptides that have the same or similar binding motifs. Two peptide sequence vectors that are far apart within the embedded space may correspond to peptides that have dissimilar binding motifs.

Model 124 is trained using metric learning via loss evaluator 125. Loss evaluator 125 may use metric learning algorithm 126 to tune the parameters of model 124. Loss evaluator 125 is used to train model 124 such that peptide sequences (and thereby, the corresponding peptides) that are of a same class are mapped closer together within the embedded space and peptide sequences (and thereby, the corresponding peptides) that are of different classes are mapped further apart within the embedded space. The class of a peptide sequence may be, for example, the individual MHC allele that is expected to (or predicted to) present the peptide corresponding to the peptide sequence. Thus, model 124 is trained such that peptide sequences that are presented by a same MHC allele are embedded closer together within the n-dimensional space as compared to peptide sequences that are presented by different MHC alleles.

The MHC gene family is divided into three subgroups: MHC class I, MHC class II, and MHC class III. The genes in these subgroups may be highly polymorphic and may each include thousands of various individual MHC alleles that are each identifiable via an allele identifier. Two different MHC alleles may both be MHC class I, may both be MHC class II, or may include a first allele of MHC class I and a second allele of MHC class II. Thus, two peptide sequences belong to different classes for the purposes of metric learning algorithm 126 when they correspond to peptides that are presented by or expected to be presented by different MHC alleles having different allele identifiers (regardless of whether these MHC alleles are both MHC class I, both MHC class II, or of MHC class I and MHC class II).

Thus, whether an MHC allele that presents a first peptide is the same as an MHC allele that presents a second peptide may be determined by the allele identifier of the MHC allele. Each MHC allele is identifiable by an allele identifier that is made up of some number of digits (e.g., 4, 6, 8, or some other number of digits). When dealing with an MHC allele in the human (i.e., an HLA allele), the allele identifier may be comprised of various letter and/or digits that form one or more fields for representing different pieces of information about the allele. The allele identifier may include one or more letters that indicate the corresponding MHC (HLA) gene, a level of expression, or both.

The allele identifier for an HLA allele may include a 4-digit, 6-digit, or 8-digit identifier for the HLA allele. In a 4-digit identifier, the first and second digits identify the allele group; the third and fourth digits identify the specific allele protein. The specific allele protein is determined based on DNA sequence and differences within the amino acid sequence of the encoded protein. A 6-digit identifier adds, to a 4-digit identifier, fifth and sixth digits that identify exon region information. Exon region information captures changes in one or more exon regions of the HLA allele such as, for example, synonymous nucleotide substitutions. An 8-digit allele identifier adds, to a 6-digit identifier as described above, seventh and eighth digits that identify intron region information. The intron region information captures changes in one or more intron regions of the HLA allele such as, for example, polymorphisms in the intron regions.

Metric learning algorithm 126 may include a set of loss functions 128 that is used to tune the parameters of model 124. Set of loss functions 128 may include, for example, without limitation, at least one of a contrastive loss function, a triplet loss function, a quadruplet loss function, a circle loss function, a multi-class n-pair loss function, a lifted structure loss function, an angular loss function, a divergence loss function, a constellation loss function, or another type of loss function that relies on learned distance metrics. A distance metric may be, for example, Euclidean distance, cosine distance, or some other type of distance metric.

Metric learning algorithm 126 uses set of loss functions 128 to train model 124 such that the peptide sequence vectors for peptides sequences that are of a same class (i.e., correspond to peptides that are presented by a same MHC allele with respect to their allele identifiers) are brought closer together in the embedded space and the peptide sequence vectors for peptide sequences that are of a different class (i.e., correspond to peptides that are presented by different MHC alleles with respect to their allele identifiers) are moved further apart from each other in the embedded space. Examples of the different types of loss functions that can be included in metric learning algorithm 126 are described in greater detail in Section III.C below.

In one or more embodiments, model 124 is trained using training peptide sequence data 130 and training allele presentation data 132. In one or more embodiments, training peptide sequence data 130 includes data (e.g., may include peptide sequence data 116) generated based on the processing of set of samples 118. In other embodiments, training peptide sequence data 130 includes other data generated from the processing of a different set of samples, which may be different with respect to at least one sample. In one or more embodiments, training peptide sequence data 130 may include one or more of peptide sequences 122, one or more other peptide sequences, or a combination thereof.

Training allele presentation data 132 includes information about which MHC alleles are expected to (or predicted to) present which peptides. For example, training allele presentation data 132 may identify a plurality of MHC (e.g., HLA) alleles that have are expected to present various peptides. An MHC allele that is expected to present a given peptide is one that has been confirmed as presenting the peptide, is predicted to present the peptide, or has otherwise been determined to be capable of presenting the peptide (e.g., based on the binding pocket of the MHC allele). In one example, for a given peptide sequence, training allele presentation data 132 identifies the one or more MHC alleles that have a binding pocket configured to bind with a binding motif present in the peptide sequence. In one or more embodiments, training allele presentation data 132 identifies a set of MHC alleles for each peptide sequence (or training peptide sequence) in training peptide sequence data 130. A set of MHC alleles includes one or more MHC alleles for each corresponding peptide sequence. In some embodiments, training allele presentation data 132 identifies, for a given peptide sequence, a single MHC allele that is expected to present the peptide (or training peptide) that corresponds to the given peptide sequence.

Training allele presentation data 132 may be generated based on, for example, without limitation, training peptide sequence data 130. For example, data analyzer 111 may include presentation model 134 that outputs training allele presentation data 132 based on input peptide sequence data (e.g., training peptide sequence data 130). In one or more embodiments, presentation model 134 may be implemented within sequence analyzer 112 (e.g., separate from or within model 124). As one example, presentation model 134 may be implemented as a separate model that receives training peptide sequence data 130 and outputs training allele presentation data 132.

Presentation model 134 may take the form of a machine learning model. For example, presentation model 134 may include a deep learning model (e.g., one or more neural networks). In one or more embodiments, presentation model 134 includes a softmax function. Presentation model 134 may be implemented using, for example, NetMHC (e.g., NetMHC 4.0, NetMHCpan, etc.). In other embodiments, presentation model 134 may be integrated with and trained concurrently with model 124 to identify, for a given peptide sequence, a set of MHC alleles that is expected (or predicted) to present the peptide sequence. For example, model 124 may be trained to generate, for a given peptide sequence, a peptide sequence vector in the embedded space and to predict a set of MHC alleles for presenting the corresponding peptide.

In one or more embodiments, presentation model 134 receives a peptide sequence as input and outputs a set of allele identifiers that identifies the respective set of MHC alleles expected (or predicted) to present the peptide corresponding to the peptide sequence.

In one or more embodiments, data analyzer 111 may retrieve, access, or otherwise obtain training allele presentation data 132 from data store 104, one or more other types of storage (e.g., database, server, cloud storage, etc.), another source, or a combination thereof. In this manner, training allele presentation data 132 may be previously generated data.

Further, in one or more embodiments, loss evaluator 125 includes miner 135 (also referred to as sampler 135), which may be implemented using hardware, software, firmware, or a combination thereof. Miner 135 may be used to implement a mining strategy (or sampling strategy) for evaluating loss during training. The mining strategy is selected to help prevent a freeze in the training of model 124 and/or to help move model 124 towards convergence more quickly. Such mining strategies are described in greater detail in Section III.D below.

After training, model 124 may be used in a prediction mode to generate a plurality of peptide sequence vectors 136 for peptide sequences 122. Each of peptide sequence vectors 136 may be a vector in the embedded space (e.g., an n-dimensional space). Model 124 is capable of generating these peptide sequence vectors 136 in a manner agnostic to peptide sequence length. The positions of peptide sequence vectors 136 relative to each other within the embedded space provide an indication of the similarity and/or dissimilarity of the corresponding peptides to each other.

Data analyzer 111 may generate output 140 based on peptide sequence vectors 136. Output 140 may include peptide sequence vectors 136, information generated using peptide sequence vectors 136, or both. Output 140 may be generated in various forms such as, for example, but not limited to, a visual representation, a spreadsheet, a list, and/or some other type of output. In some embodiments, output 140 includes a listing of the peptide sequence vectors 136. In some embodiments, output 140 includes a spreadsheet identifying the peptide sequence vectors 136 as well as other information (e.g., the corresponding peptide sequence for each peptide sequence vector, the MHC allele predicted to present the corresponding peptide, other information, or a combination thereof).

In one or more embodiments, output 140 includes a visual (e.g., graphical) representation of peptide sequence vectors 136 in a k-dimensional space. The k-dimensional space may include the same or fewer dimensions than the n-dimensional space of peptide sequence vectors 136 (i.e., k≤n). As one example, peptide sequence vectors 136 corresponding to peptides 120 may be vectors having 32 dimensions, while the graphical representation may show peptides 120 represented in a 2-dimensional or 3-dimensional space. Presenting peptide sequence vectors 136 in a reduced dimensional space may allow a human to more quickly and/or easily understand or ascertain the similarity relationships between the corresponding peptides.

Output 140 may, in some cases, classify peptides 120 in clusters based on the positions of peptide sequence vectors 136 within the embedded space. For example, each of peptides 120 may be assigned to a different cluster (or group or category) based on its corresponding peptide sequence vector's position within the n-dimensional space. In one or more embodiments, the peptide sequence vectors (and thereby, peptide sequences and corresponding peptides) assigned to a same cluster (or group or category) may generally have a same binding motif or a set of similar binding motifs. Data analyzer 111 may use one or more clustering algorithms to identify these clusters. Such clustering algorithms include, but are not limited to, a K-means clustering algorithm, an affinity propagation clustering algorithm, an agglomerative clustering algorithm, a mini-batch K-means clustering algorithm, a mean shift clustering algorithm, a spectral clustering algorithm, a Gaussian mixture clustering algorithm, a Balanced Iterative Reducing and Clustering (BIRCH) algorithm, a Density-Based Spatial Clustering of Applications and Noise (DBSCAN) clustering algorithm, and an Ordering Points to Identify the Clustering Structure (OPTICS) algorithm.

In one or more embodiments, output 140 is sent to candidate selector 114 for processing. Candidate selector 114 may include a model (e.g., a machine learning model or another type of model) that is trained to select diverse group of candidate peptides 110 for development of peptide therapeutic 108 based on output 140. For example, candidate selector 114 may use output 140 to rank and select a top x number of candidate peptides for inclusion in diverse group of candidate peptides 110. The x number of candidate peptides may be, for example, but is not limited to, 2, 3, 4, 5, 6, 7, 8, 9, 10, 11, 12, 13, 14, 15, 16, 17, 18, 19, 20, 21, 22, 23, 24, 25, 26, 27, 28, 29, 30 or some other number of candidate peptides.

Candidate selector 114 may select the candidate peptides that form diverse group of candidate peptides 110 to ensure that dissimilar candidate peptides are selected for peptide therapeutic 108 to improve the chances of MHC presentation and the triggering of an immune response. Candidate selector 114 may select diverse group of candidate peptides 110 such that the included peptides are diverse with respect to, for example, binding motifs and/or the MHC alleles that present the peptides.

The candidate peptides may be selected in various ways. Candidate selector 114 may, for example, identify a plurality of clusters of the peptide sequence vectors 136 using output 140. Output 140 may expressly identify these clusters or candidate selector 114 may use the information in output 140 and one or more clustering algorithms, as described above, to identify these clusters. Candidate selector 114 may then select, for example, at least one peptide sequence vector and thereby its corresponding peptide from each of the different clusters to ensure sufficient diversity between the candidate peptides. For example, for a given cluster, candidate selector 114 may select a peptide that is nearest a center of that cluster. The center may be a centroid (e.g., a Euclidean distance-based centroid), a mean center, a median center, a density-based center, or another type of center.

In some examples, candidate selector 114 identifies a subset of the clusters that are greater than a selected threshold distance from each other (e.g., with respect to their centers) and then selects at least one peptide from each cluster in this subset. For example, each cluster in the subset may have a distance greater than the threshold from every other cluster in the subset.

When multiple peptides are selected from a given cluster, candidate selector 114 may select these peptides in different ways to ensure local diversity. As one example, candidate selector 114 may identify a center (e.g., centroid, mean center, median center, density-based center, etc.) of the selected cluster and then select two or more peptides that are halfway between the center and an edge of the cluster, while also maximizing a distance between the two or more peptides. In another example, candidate selector 114 may identify two or more peptides along (e.g., at or near) the edge of the selected cluster while also maximizing the distances between the two or more peptides. In yet another example, candidate selector 114 may use density estimation and/or computations to select multiple peptides from a selected cluster. For example, candidate selector 114 may use density estimation to identify multiple “local” centers (e.g., centroids) within a given cluster and may then select the peptide nearest each of these local centers.

In one or more embodiments, candidate selector 114 uses an algorithm to select an X (e.g., 4, 5, 8, 10, 12, 15, 20, 25, 30, 50, 75, etc.) number of peptides such that the distance between each possible pairing of the X number of peptides is maximized. In some cases, candidate selector 114 selects the X number of peptides such that the distance between any two peptides of the X number of peptides is greater than a distance threshold. This type of selection ensures that a minimum level of dissimilarity between the peptides.

Candidate selector 114 may use one or more filters to reduce the pool of peptides from which the candidate peptides are selected. For example, candidate selector 114 may reduce the pool of peptides to highly rated/ranked peptides that have well-known or clearly defined binding motifs. Candidate selector 114 may use one or more filters to reduce “noise” in such that the clusters may be more well-defined.

In one or more embodiments, the candidate peptides of diverse group of candidate peptides 110 may be, for example, mutant peptides. For example, the candidate peptides may have peptides that have peptide sequences that include variant-coding sequences.

In one or more embodiments, data analyzer 111 generates report 142 based on output 140. Report 142 may be generated by data analyzer 111 using information output from sequence analyzer 112, information output from candidate selector 114, or both. For example, report 142 may include at least a portion of output 140, an identification of diverse group of candidate peptides 110, or both. An identification of diverse group of candidate peptides 110 may include, for example, an identification of the peptide sequences that correspond to diverse group of candidate peptides 110. In some embodiments, report 142 may include the exact output of model 124, a transformed or filtered version of output 140, or both. In some cases, data analyzer 111 may generate notifications, recommendations, alerts, or other information based on at least a portion of output 140, the identification of group of candidate peptides 110, or both, with this additional information being included in report 142. In some embodiments, report 142 may be generated by sequence analyzer 112 and/or candidate selector 114.

Report 142 may include, for example, a recommendation regarding which candidate peptides of diverse group of candidate peptides 110 to select for inclusion in peptide therapeutic 108. For example, report 142 may identify a plurality of treatment peptides 144 for inclusion in peptide therapeutic 108. Treatment peptides 144 includes two or more peptides selected from diverse group of candidate peptides 110. Further, treatment peptides 144 includes at least two peptides that are dissimilar (e.g., dissimilar with respect to binding motifs). In one or more embodiments, peptide therapeutic 108 is developed to include treatment peptides 144, precursors for treatment peptides 144, or one or more nucleic acids (e.g., DNA and/or RNA) that encode treatment peptides 144 or precursors of treatment peptides 144). Report 142 may identify treatment peptides 144, the precursors for treatment peptides 144, or the one or more nucleic acids (e.g., DNA and/or RNA) that encode the treatment peptides 144 or precursors thereof.

Report 142 may include, for example, instructions for facilitating the manufacture of peptide therapeutic 108 based on the recommended candidate peptides. In one or more embodiments, report 142 may include an alert for triggering a computerized process involved in the manufacture of peptide therapeutic 108.

Report 142 may be displayed on graphical user interface 150 on display system 106 in one or more embodiments. A user may, for example, view report 142 and/or interact with report 142 via graphical user interface 150 and use report 142 to make decisions about the design and development of peptide therapeutic 108. In some embodiments, data analyzer 111 sends report 142 to remote system 152 (e.g., wirelessly). Remote system 152 may be a cloud computing platform, cloud storage, another computer system, a user device (e.g., a smartphone, a tablet, a laptop, etc.) or some other type of platform. In some embodiments, remote system 152 may be a treatment manufacturing system (or machine) or a portion thereof that can use report 142 to manufacture peptide therapeutic 108.

### II.B. Example of a Machine Learning Model—Attention-Based

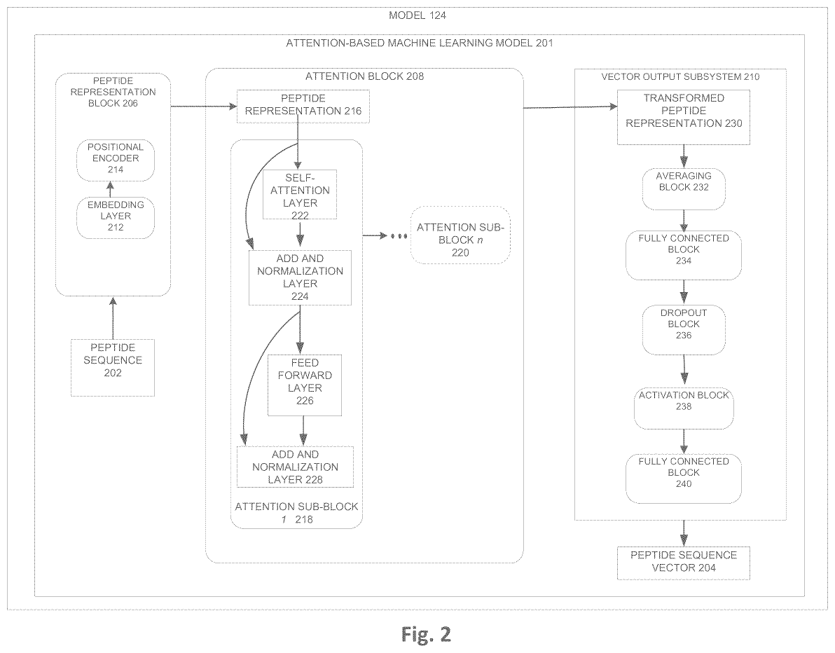

FIG. 2 is a schematic diagram of one example of a configuration for model 124 in FIG. 1 in accordance with one or more embodiments. Model 124 may be configured to receive peptide sequence 202 as input and generate peptide sequence vector 204 as output. Peptide sequence 202 may be one example of an implementation for one of the peptide sequences 122 described in FIG. 1 or for one of the peptide sequences in training peptide sequence data 130 in FIG. 1. Peptide sequence vector 204 may be one example of an implementation for one of the peptide sequence vectors 136 described in FIG. 1.

In one or more embodiments, model 124 takes the form of attention-based machine learning model 201. In one or more embodiments, attention-based machine learning model 201 may be implemented in a manner as described in U.S. Publication No. 2022/0122690 or International Publication No. WO2022016125, each of which is incorporated by reference in its entirety.

In one or more embodiments, attention-based machine learning model 201 includes peptide representation block 206, attention block 208, and vector output subsystem 210. Each of peptide representation block 206, attention block 208, and vector output subsystem 210 may include one or more sub-blocks and/or layers. A sub-block may be comprised of any number of layers (or units).

Peptide representation block 206 may include at least one embedding layer 212 and may optionally include, for example, positional encoder 214. Embedding layer 212 receives peptide sequence 202 as input and embeds peptide sequence 202 to generate an embedded peptide representation. Peptide sequence 202 may be embedded by, for example, transforming peptide sequence 202, which is a non-numeric representation (e.g., a string of amino-acid identifiers, a string of nucleic acid identifiers), into a numeric representation that becomes the embedded peptide representation. The embedding can be performed using, for example, one-hot encoding, evolutionarily-motivated encodings such as BLOcks Substitution Matrix (BLOSUM), randomly or pseudorandomly initialized learned embeddings, or a combination thereof.

In some cases, various attention mechanisms may be unable to detect potential information conveyed by an order of values in an input data set, such as peptide sequence 202. Accordingly, a positional encoder(s) such as positional encoder 214 may be used and applied to the embedded peptide representation generated by embedding layer 212. Position encoder 214 may perform positional encoding using an encoding algorithm that is learned or fixed. Positional encoder 214 receives the embedded peptide representation from embedding layer 212 and positionally encodes the embedded peptide representation to generate peptide representation 216 that represents the peptide sequence.

Peptide representation 216 may, for example, a multi-dimensional vector that represents or otherwise corresponds to each peptide element (e.g., each amino acid, nucleic acid, codon, etc.) of peptide sequence 202. For example, peptide representation 216 may be a matrix that includes a vector (e.g., having between 20 to 1000 dimensions) for each peptide element of peptide sequence 202. Thus, for a peptide sequence comprising e peptide elements (e.g., amino acids, nucleic acids, or codons), the matrix may include e vectors having d dimensions (e.g., 20≤d≤1000).

In one or more embodiments, a fixed positional encoding may be defined using a sine and/or cosine function (e.g., having an intra-sequence position and/or a dimension as the independent variables). The positional encoding output by position encoder 214 may have a same dimension as the embedded peptide representation output by embedding layer 212. In one or more embodiments, the positional encodings may be summed with the embedded representation to produce a position-indicative embedded representation of the sequence. Accordingly, in or more embodiments, peptide representation 216 generated by peptide representation block 206 may be the encoded representation of or an aggregation (e.g., concatenation or sum) of the encoded representation formed by positional encoder 214 and the embedded peptide representation formed by embedding layer 212.

In other embodiments, positional encoder 214 may generate a unique learned embedding for each possible position in the peptide sequence. These embeddings are then added to the embedded peptide representation formed by embedding layer 212 to form peptide representation 216.

Peptide representation 216 is sent as input into attention block 208. Attention block 208 may include one or more sub-blocks and/or layers. For example, attention block 208 may include attention sub-block 1 218 and, optionally, one or more other attention sub-blocks up to attention sub-block n 220. When multiple attention sub-blocks are present in attention block 208, these attention sub-blocks may be connected serially (e.g., daisy-chained together to produce a final output). In one or more embodiments, an attention block 208 may use a set of query weights, a set of key weights, and a set of value weights to determine, for a given peptide element (e.g., amino acid) of a peptide sequence, an extent to which each of one or more other peptide elements are to be “attended to” when processing the given peptide element.

Attention sub-block 1 218 may be implemented in various ways. In one or more embodiments, attention sub-block 1 218 includes, but is not limited to, self-attention layer 222, add and normalization layer 224, feed forward layer 226, and add and normalization layer 228. With this configuration for attention sub-block 1 218, attention sub-block 1 218 may also be referred as a transformer encoder. If present, the one or more other attention sub-blocks in attention block 208 up to attention sub-block n 220 may be implemented in a manner similar to attention sub-block 1 218.

Self-attention layer 222 may be implemented using, for example, a one-head attention unit or a multi-head attention unit. Self-attention layer 222 transforms peptide representation 216 into a transformed representation. In add and normalization layer 224, the transformed representation may be added to the position-indicative embedded representation of the sequence (e.g., peptide representation 216) via, for example, a residual connection, and the summed representation can be normalized.

The normalized data can be fed to the corresponding feed forward layer 226 (e.g., a fully connected feedforward network). Feed forward layer 226 can affect (for example), for each position, one, two, three, or more linear transformations and/or may include an activation (e.g., a ReLU activation) between each of the linear transformations. For example, feed forward layer 226 can be represented by:

$$FF ( x ) = max ( 0 , {xW}_{1} + {b}_{1} ) {W}_{2} + {b}_{2} , \qquad ( 1 )$$

where x is an input to the layer, W₁ and W₂ are slopes of the linear transformations and b₁ and b₂ are intercepts of the linear transformation. A dimensionality of an output of a particular attention sub-block's feed forward layer may be the same as a dimensionality of an input to the attention sub-block's feed forward layer. Thus, in some instances, to preserve representations of various types of information, the input and output can be summed and normalized (e.g., via another residual connection through another add and normalization layer such as add and normalization layer 228).

Attention block 208 receives and processes peptide representation 216 using the set of attention sub-blocks to generate transformed peptide representation 230 as output. Transformed peptide representation 230 may be a matrix that includes a vector (e.g., having between 20 to 1000 dimensions) for each peptide element of peptide sequence 202. Transformed peptide representation 230 may be sent into vector output subsystem 210 for processing.

Vector output subsystem 210 may include various blocks, sub-blocks, layers, or combination thereof for generating the final output of model 124, which includes peptide sequence vector 204. In one or more embodiments, vector output subsystem 210 includes averaging block 232, fully connected block 234, dropout block 236, activation block 238, and fully connected block 240. Each of fully connected block 234 and fully connected block 240 may include, for example, one or more fully connected layers. Dropout block 236 may include, for example, one or more dropout layers. Activation block 238 may include one or more activation layers (e.g., linear or nonlinear functions) such as, for example, a rectified linear function, which is a nonlinear function.

Transformed peptide representation 230 may be further processed before information is sent into fully connected block 234. In one or more embodiments, averaging block 232 is used to average the various vectors in transformed peptide representation 230. For example, the vectors in transformed peptide representation 230 for the different peptide elements of peptide sequence 202 may be averaged together to form an averaged representation of peptide sequence 202 that includes a single numerical value for each peptide element (e.g., amino acid, nucleic acid, or codon) of peptide sequence 202. Thus, the averaged representation may be a single vector. This averaged representation may be sent as input into fully connected block 234.

In some embodiments, averaging block 232 may be replaced by a block that concatenates the different vectors together to form an aggregate. This block may then add a Beginning of Sequence (BoS) token in front of the aggregate to form a new representation that is sent into fully connected block 234.

In some embodiments, fully connected block 234 is configured to output a vector that has fewer dimensions than the averaged representation fed as input into fully connected block 234. Fully connected block 234 may include, for example, a single hidden layer, two hidden layers, or three or more hidden layers. A number of nodes in an initial hidden layer may be larger than a number of nodes in a subsequent hidden layer. For example, a first hidden layer can include 256 nodes, while a second hidden layer can include 126 nodes.

Dropout block 236 may be used to apply dropout regularization to one or more layers of fully connected block 234. In particular, dropout block 236 may be used to disable some portion of the neurons in one or more of the hidden layers in fully connected block 234.

Activation block 238 may be used to apply a nonlinear activation function to fully connected block 234. For example, activation block 238 may use one or more rectified linear units (ReLU) to convert any negative values into zeros.

Fully connected block 240 may receive, as input, the output generated after the application of dropout regularization and the activation function on fully connected block 234 and generate an output that is peptide sequence vector 204. Similar to fully connected block 234, fully connected block 240 may be configured to output a vector that has fewer dimensions than the input fed into fully connected block 240. Fully connected block 240 may include, for example, a single hidden layer, two hidden layers, or three or more hidden layers. A number of nodes in an initial hidden layer may be larger than a number of nodes in a subsequent hidden layer.

Peptide sequence vector 204 may be a vector having n dimensions. In some instances, the n number of dimensions may be selected such that the training (or learning) of model 124 is sufficiently robust and rich. For example, in some instances, it may be desirable for model 124 to generate peptide sequence vector 204 in a lower dimensional (e.g., n is less than 250) space to encourage more robust or substantive learning by model 124. In one or more embodiments, peptide sequence vector 204 is a vector in an n-dimensional space having 16 dimensions, 32 dimensions, 64 dimensions, 128 dimensions, or some other number of dimensions.

Peptide sequence vector 204 captures, represents, or otherwise provides information about peptide sequence 202. For example, peptide sequence vector 204 may capture information about the peptide sequence 202 such that peptide sequence vector 204 relative to another peptide sequence vector for another peptide sequence provides an indication of a similarity or dissimilarity of the two peptide sequences.

The configuration of attention-based machine learning model 201 in FIG. 2 is but one example of an implementation for attention-based machine learning model 201 (and thereby model 124). In other embodiments, other configurations for attention-based machine learning model 201 may be used. For example, in one or more embodiments, vector output subsystem 210 may include one or more other layers for filtering, selecting, transforming, or otherwise modifying the output of any one or more of the blocks or layers in vector output subsystem 210 to ultimately generate peptide sequence vector 204.

### III. Example of Methodologies Used in Selection of Diverse Peptides for a Peptide Therapeutic

### III.A. Selecting a Diverse Group of Candidate Peptides Using a Machine Learning Model

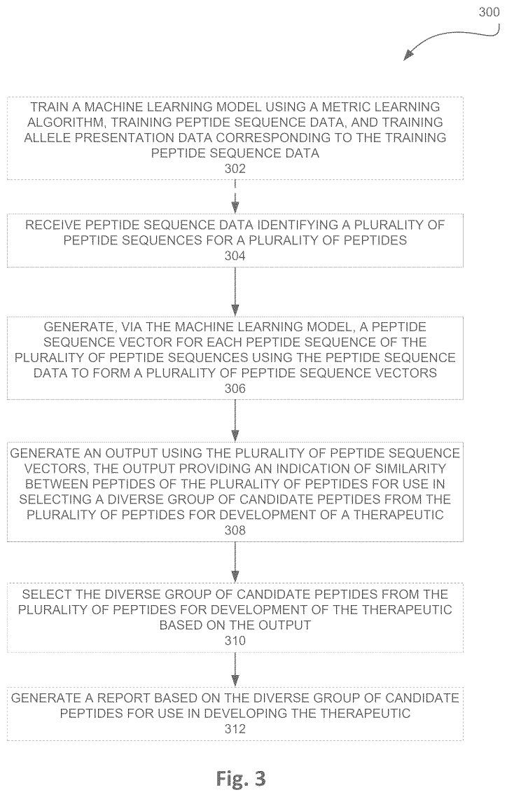

FIG. 3 is a flowchart of a process for use in developing a therapeutic in accordance with one or more embodiments. Process 300 may be implemented using, for example, therapeutic development system 100 described with respect to FIG. 1. Process 300 may be implemented for use in the development of a peptide therapeutic. For example, process 300 may be used to select a diverse group of candidate peptides, such as the diverse group of candidate peptides 110 described with respect to FIG. 1, for use in the development of a peptide therapeutic, such as peptide therapeutic 108 in FIG. 1.

Process 300 may include step 302. Step 302 includes training a machine learning model using a metric learning algorithm, training peptide sequence data, and training allele presentation data corresponding to the training peptide sequence data. The machine learning model may be trained to generate a peptide sequence vector (or embedding) for a given peptide sequence. The peptide sequence may take the form of, for example, an amino acid sequence, a nucleic acid sequence, a codon sequence, or another type of sequence that defines at least a portion of a corresponding peptide. The machine learning model may be, for example, model 124 in FIG. 1 or FIG. 2. The machine learning model may be, for example, attention-based machine learning model 201 in FIG. 2. The machine learning model may be, for example, a deep learning model and may include, without limitation, one or more neural networks.

The training peptide sequence data may be, for example, training peptide sequence data 130 in FIG. 1. The training peptide sequence data includes peptide sequences for training (which may be also referred to as training peptide sequences).

The training allele presentation data used in the training of the machine learning model may identify, for each peptide sequence of the training peptide sequence data, one or more MHC alleles that have a binding pocket (or binding groove) configured to bind with a binding motif in the peptide sequence. The training allele presentation data may be, for example, training allele presentation data 132 in FIG. 1.

In one or more embodiments, the training allele presentation data is generated independently of the training in step 302. For example, the training allele presentation data may be generated by another model (e.g., presentation model 134 in FIG. 1) prior to the training in step 302. The training allele presentation data may then be stored for later use in step 302. In some instances, the training allele presentation data is stored in a data store (e.g., data store 104 in FIG. 1) or some other type of data storage or source.

In other embodiments, the training allele presentation data is generated as part of the training in step 302. For example, the machine learning model may include a first system for generating a peptide sequence vector for a given peptide sequence and a second system for identifying a set of MHC alleles predicted to present the given peptide sequence. The second subsystem may be trained prior to the first subsystem being trained.

The metric learning algorithm used in the training of the machine learning model may be, for example, metric learning algorithm 126 in FIG. 1. The metric learning algorithm includes one or more loss functions that are used to evaluate learned distance metrics for the peptide sequence vectors for various groups of peptide sequences (e.g., 2, 3, 4, or more peptide sequences) to affect how the parameters (e.g., weights) of the machine learning model are adjusted after each training batch and/or after each epoch. For example, a learned distance metric between a pair of peptide sequence vectors may be Euclidean distance, cosine distance, or some other type of distance metric. In one or more embodiments, the metric learning algorithm includes at least one of a contrastive loss function, a triplet loss function, a quadruplet loss function, a circle loss function, a multi-class n-pair loss function, a lifted structure loss function, an angular loss function, a divergence loss function, a constellation loss function, or another type of loss function that relies on learned distance metrics.

The training allele presentation data is used to determine how the training of machine learning model via the metric learning algorithm learning is implemented. For example, in one or more embodiments, the metric learning algorithm may use the training allele presentation data to train the machine learning model to generate the peptide sequence vectors for peptides that are presented by a same MHC allele closer together in the n-dimensional space and to generate peptide sequence vectors for peptides that are presented by different MHC alleles further away from each other in the n-dimensional space. Examples of methodologies that may be used for training are described in Section III.B below.

Step 304 of process 300 includes receiving peptide sequence data identifying a plurality of peptide sequences for a plurality of peptides. The peptide sequence data may be, for example, peptide sequence data 116 in FIG. 1. The peptide sequence data may be formed by, for example, at least a portion of the training sequence data. In other embodiments, the peptide sequence data may be data generated from the processing of a set of samples (e.g., set of samples 118 in FIG. 1). In one or more embodiments, the peptide sequence data may include peptide sequences that are also included in training sequence data. In one or more embodiments, the peptide sequence data may include peptide sequences that were not included in the training peptide sequence data. The peptide sequences for the peptides may be, for example, the peptide sequences 122 for the peptides 120 in FIG. 1. Peptide sequence 202 in FIG. 2 may be one example of one of the plurality of peptide sequences in step 304.

Step 306 includes generating, via the machine learning model, a plurality of peptide sequence vectors in an n-dimensional space for respective ones of the plurality of peptide sequences and thereby, for respective ones of the plurality of peptides. The machine learning model has been trained, via step 302, using the metric learning algorithm, the training peptide sequence data, and the training allele presentation data corresponding to the training peptide sequence data. The training peptide sequence data identifies a training peptide sequence that corresponds to each training peptide of a plurality of training peptides. The training allele presentation data identifies, for each training peptide sequence in the training peptide sequence data, a set of major histocompatibility complex (MHC) alleles expected to present the training peptide that corresponds to the training peptide sequence.

The metric learning algorithm is used to train the machine learning model such that peptide sequence vectors for peptides that are presented by a same MHC allele are embedded closer together in the n-dimensional space and peptide sequence vectors for peptides that are presented by different MHC alleles are embedded further away from each other in the n-dimensional space. Thus, the position of any peptide sequence vector with respect to the n-dimensional space may provide an indication of its similarity or dissimilarity to other peptide sequence vectors in the n-dimensional space, and thereby their corresponding peptides.

For example, the metric learning algorithm is used to train the machine learning model such that a first distance between a first pair of the peptide sequence vectors generated within the n-dimensional space for a first respective pair of the peptides that are presented by a same MHC allele is less than a second distance between a second pair of the peptide sequence vectors generated within the n-dimensional space for a second pair of the peptides that are presented by different MHC alleles. The first pair of the peptide sequence vectors and the second pair of the peptide sequence vectors may include a common peptide sequence vector or may include wholly different peptide sequence vectors. For example, the first pair may include peptide sequence vector A (PSV-A) and PSV-B, while the second pair may include PSV-A and PSV-C. In other examples, the first pair includes PSV-A and PSV-B, while the second pair includes PSV-C and PSV-D.

In one or more embodiments, step 306 includes converting each peptide sequence (e.g., peptide sequence 202 in FIG. 2) into a peptide representation (e.g., peptide representation 216 in FIG. 2). The peptide representation may include multiple vectors with a vector for each peptide element (e.g., amino acid, nucleic acid, or codon) of the peptide sequence. Each vector in the peptide representation may include e elements (e.g., 20≤e≤1000). Step 306 further includes converting each peptide representation into a peptide sequence vector (e.g., peptide sequence vector 204 in FIG. 2). In one or more embodiments, the n elements of each of the peptide sequence vectors include fewer elements than the e elements of the vectors of the peptide representation. In some cases, however, the n elements may include more elements than the peptide elements that make up the original peptide sequence.

Step 308 includes generating an output using the plurality of peptide sequence vectors, the output providing an indication of similarity between peptides of the plurality of peptides for use in selecting a diverse group of candidate peptides from the plurality of peptides for development of the therapeutic. In one or more embodiments, the output may be, for example, output 140 in FIG. 1. The output may include the plurality of peptide sequence vectors, information generated based on the plurality of peptide sequence vectors, or both.

The output may include, for example, but is not limited to, a visual (e.g., graphical) representation of the peptide sequence vectors in a k-dimensional space. The k-dimensional space may include the same or fewer dimensions than the n-dimensional space of the peptide sequence vectors. As one example, the peptide sequence vectors of the peptides may be vectors having 32 dimensions, while the graphical representation may show the peptides represented in a 2-dimensional or 3-dimensional space. This reduction in dimensions may enable a human to more readily comprehend and quickly ascertain the distance relationships between the different peptide sequence vectors.

In one or more embodiments, the output may provide a classification of the peptides in clusters. For example, each of the peptides may be assigned to a different cluster (or group or category) based on its position within the n-dimensional space relative to the positions of the other peptides. Each cluster (or group or category) may generally correspond to a unique binding motif or a set of similar binding motifs.

Process 300 may optionally include, in one or more embodiments, step 310. Step 310 includes selecting a diverse group of candidate peptides from the plurality of peptides for development of the therapeutic based on the output. For example, the output generated in step 306 may be used to select a diverse group of candidate peptides (e.g., diverse group of candidate peptides 110 in FIG. 1) for the development of a peptide therapeutic (e.g., peptide therapeutic 108 in FIG. 1). The diverse group of candidate peptides may include at least two dissimilar candidate peptides. The peptide therapeutic may be, for example, a peptide vaccine.

The diverse group of candidate peptides may be selected such that the included peptides are diverse with respect to, for example, but not limited to, binding motifs. For example, the diverse group of candidate peptides may include at least two candidate peptides that have dissimilar binding motifs. In some instances, the selection in step 310 may be performed such that that a bias towards any single binding motif is reduced.

In one or more embodiments, the output may be used to rank and select a top x number of candidate peptides for inclusion in the diverse group of candidate peptides for the development of a peptide vaccine (e.g., neoantigen vaccine). The x number of candidate peptides may be, for example, but is not limited to, 2, 3, 4, 5, 6, 7, 8, 9, 10, 11, 12, 13, 14, 15, 16, 17, 18, 19, 20, or some other number of candidate peptides.

Selecting the diverse group of candidate peptides in step 310 may be performed in various ways. In one or more embodiments, when the output generated in step 308 identifies or presents an indication of clusters that correspond to different binding motifs and/or different sets of similar binding motifs, one, two, or three peptides may be selected from each cluster to ensure binding motif diversity. For example, a peptide nearest the center of each cluster may be selected as a candidate peptide. The center of the cluster may be a centroid (e.g., a Euclidean distance-based centroid), a mean center, a median center, a density-based center, or a different type of center of the cluster.

In other embodiments, two or more peptides may be selected such that those peptides are the most dissimilar peptides. For example, the output generated in step 308 may provide a ranking of pairs of the peptides based on distance. The diverse group of candidate peptides may then be selected by choosing some number (e.g., 1, 2, 3, 4, etc.) of the pairs of peptides that have the greatest distance between each other.

In still other embodiments, when output takes the form of a visual representation in a k-dimensional space, step 310 may be performed by dividing the k-dimensional space into a plurality of regions and selecting one or more candidate peptides from each of the plurality of regions. In one example, the k-dimensional space may be a 2-dimensional or 3-dimensional space that is divided into quadrants with one or more candidate peptides being selected from each of the different quadrants. In some instances, one candidate peptide is selected from each quadrant, the candidate peptide being the furthest away from the other quadrants.

In some embodiments, the information provided by the output may be used in combination with other information to select the diverse group of candidate peptides. This other information may include, for example, quantity information identifying, for example, a quantification of each of the peptide sequences detected in a given sample. In some cases, the other information may include, for example, biological information about the subject who will be receiving the peptide therapeutic.

The different ways of selecting the diverse group of candidate peptides described above may be just some examples of the ways in which step 310 may be performed to ensure peptide (e.g., binding motif) diversity. Selecting a group of candidate peptides that has such peptide (e.g., binding motif) diversity may help improve the overall chances for presentation of the one or more peptides included in the peptide therapeutic by one or more MHC alleles in a subject. For example, having dissimilar peptides in a peptide therapeutic may help account for biological circumstances that can lead to different binding motifs actually being favored in a subject compared to what was expected. Such biological circumstances may include, for example, the microenvironment (e.g., pH, temperature, etc.) of a tumor or diseased tissue, the biological makeup of the particular subject, one or more comorbidities, etc. Thus, selecting a group of candidate peptides that has binding motif diversity may help improve the overall efficacy of the peptide therapeutic.

Process 300 may optionally include step 312. Step 312 includes generating a report based on the diverse group of candidate peptides for use in developing the therapeutic. The report may be, for example, report 142 in FIG. 1. The report may include, for example, at least a portion of the output generated in step 308, an identification of the diverse group of candidate peptides selected in step 310 (e.g., via an identification of the peptide sequences that correspond to the diverse group of candidate peptides), the peptide sequence vectors generated in step 306, or a combination thereof. In some embodiments, the report may include a transformed or filtered version of the output generated in step 308. The report may include, for example, one or more notifications, recommendations, alerts, and/or other information.

In one or more embodiments, the report may include a recommendation regarding which candidate peptides of the diverse group of candidate peptides 110 to select for inclusion in the therapeutic as the treatment peptides. The report may identify the precursors for the treatment peptides and/or a nucleic acid sequence that encodes the treatment peptides and/or the precursors thereof.

The report may include, for example, instructions for facilitating the manufacture of the therapeutic. In one or more embodiments, the report may include an alert for triggering a computerized process involved in the manufacture of the therapeutic. The report may be used to make decisions about the design and development of the therapeutic and/or to manufacture the therapeutic.

### III.B. Training the Machine Learning Model

### III.B.1. Example of a Training Methodology-General

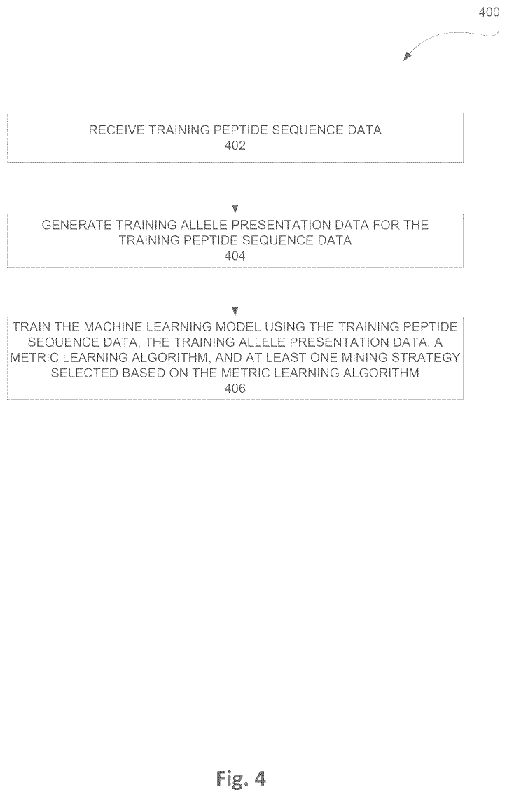

FIG. 4 is a flowchart of a process for training a machine learning model to generate peptide sequence vectors for peptide sequences in accordance with one or more embodiments. Process 400 may be implemented using, for example, therapeutic development system 100 described with respect to FIG. 1. Process 400 may be implemented to train a machine learning model such as, for example, without limitation, model 124 in FIG. 1 and/or attention-based machine learning model 201 in FIG. 2. Process 400 may be one example of an implementation for a process that may be used to perform step 302 in FIG. 3.

Step 402 includes receiving training peptide sequence data. The training peptide sequence data may be, for example, training peptide sequence data 130 in FIG. 1. The training peptide sequence data includes peptide sequences (which may be referred to as training peptide sequences), each of which forms at least a portion of a peptide. A training peptide sequence in the training peptide sequence data may take the form of, for example, an amino acid sequence, a nucleic acid sequence, a codon sequence, or another type of sequence that defines at least a portion of the corresponding peptide.

Step 404 includes generating training allele presentation data for the training peptide sequence data. The training allele presentation data may be, for example, allele presentation data 132 in FIG. 1. In some embodiments, the training allele presentation data is generated by a model such as presentation model 134 in FIG. 1.

The training allele presentation data identifies a set of MHC alleles expected to present the peptide corresponding to each peptide sequence in the training peptide sequence data. In one or more embodiments, when multiple MHC alleles are capable of presenting a given peptide having a given peptide sequence, the MHC allele most likely to present the given peptide is included in the training allele presentation data.

When dealing with training peptide sequence data for humans, the training allele presentation data includes a label for each peptide sequence of the training peptides sequence data, the label identifying an HLA allele that is predicted to present the peptide corresponding to the peptide sequence. The label may be, for example, an allele identifier that is comprised of various letter and/or digits that form one or more fields for representing different pieces of information about the HLA allele. As previously discussed in Section II.A., the allele identifier may include one or more letters that indicate the corresponding HLA gene, a level of expression, or both. The allele identifier for an HLA allele may include, for example, but is not limited to, a 4-digit, 6-digit, or 8-digit identifier for the HLA allele.

Step 406 includes training the machine learning model using the training peptide sequence data, the training allele presentation data, a metric learning algorithm, and at least one mining strategy selected based on the metric learning algorithm. The metric learning algorithm comprises at least one of a contrastive loss function, a triplet loss function, a quadruplet loss function, a circle loss function, a multi-class n-pair loss function, a lifted structure loss function, an angular loss function, a divergence loss function, or a constellation loss function. The metric learning algorithm may be, for example, metric learning algorithm 126 in FIG. 1.

The at least one mining strategy used in step 406 may be selected based on the type of loss function(s) included in the metric learning algorithm. In one or more embodiments, step 406 includes using a hard negative mining strategy, a semi-hard negative mining strategy, or both. Such mining strategies are described in greater detail below in Section III.D.

### III.B.2. Example of a Training Methodology-Batch Processing

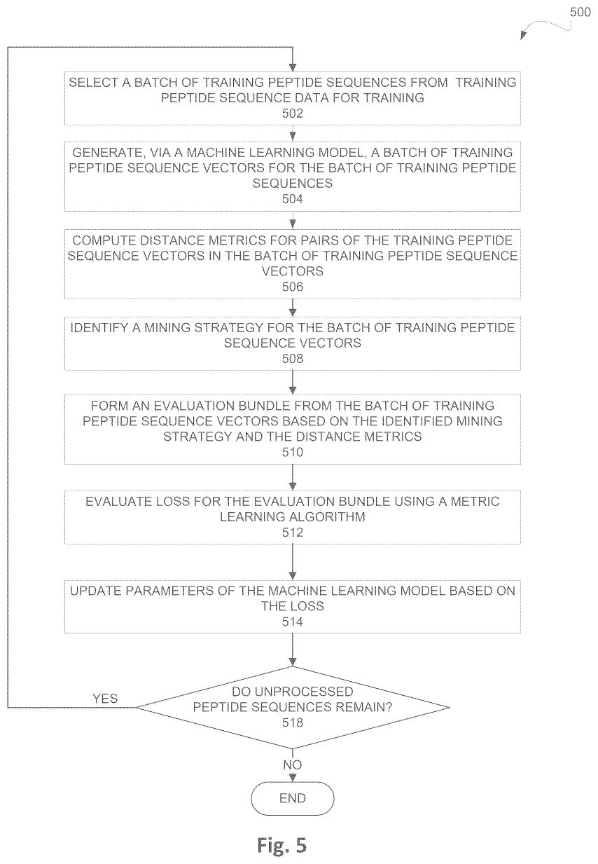

FIG. 5 is a flowchart of a process for training a machine learning model to generate peptide sequence vectors for peptide sequences in accordance with one or more embodiments. Process 500 may be implemented using, for example, therapeutic development system 100 described with respect to FIG. 1. Process 500 may be implemented to train a machine learning model such as, for example, without limitation, model 124 in FIG. 1 and/or attention-based machine learning model 201 in FIG. 2. Process 500 may be one example of a process that may be used in performing step 302 in FIG. 3. In some instances, process 500 may be one example of an implementation for a process used to perform step 406 described in FIG. 4.

Step 502 includes selecting a batch of training peptide sequences from training peptide sequence data for processing. The training peptide sequence data may be, for example, training peptide sequence data 130 in FIG. 1. The training peptide sequence data may be the training peptide sequence data received in step 402 in FIG. 4. The batch of training peptide sequences may include all of the training peptide sequences or a subset of the training peptide sequences. For example, the batch may include, but is not limited to, 5, 10, 20, 30, 50, 50, 60, 70, 80, 90, 100, 150, 200, 250, or some other number of training peptide sequences.

Step 504 includes generating, via a machine learning model, a batch of training peptide sequence vectors for the batch of training peptide sequences. The machine learning model may be, for example, model 124 in FIG. 1. The machine learning model may be, for example, attention-based machine learning model 201 in FIG. 2. The machine learning model may be, for example, a deep learning model and may include, for example, but is not limited to, one or more neural networks.

The batch of training peptide sequence vectors includes a training peptide sequence vector for each training peptide sequence in the batch of training peptide sequences. Each training peptide sequence vector in this batch of training peptide sequence vectors may be an n-dimensional vector (or embedding) that represents the corresponding training peptide sequence.

Step 506 includes computing distance metrics for pairs of the training peptide sequence vectors in the batch of training peptide sequence vectors. In one or more embodiments, step 506 includes computing a distance metric (e.g., a Euclidean distance) for each pairing of training peptide sequence vectors in the batch of training peptide sequence vectors. In this manner, a distance metric may be computed for the distance between each training peptide sequence vector and every other training peptide sequence vector.

Step 508 may include identifying a mining strategy for the batch of training peptide sequence vectors. In one or more embodiments, step 508 may be implemented by, for example, without limitation, loss evaluator 125 in FIG. 1. For example, step 508 may be implemented by miner 135 of loss evaluator 125 in FIG. 1. In step 508, identifying the mining strategy may include selecting a mining strategy from a set of mining strategies based on the batch number for the current batch of training peptide sequence vectors. The mining strategy identified in step 508 is the strategy to be used in generating groupings of training peptide sequence vectors from the current batch of training peptide sequence vectors for evaluating loss. For example, the selected mining strategy may determine which pairs, triplets, quadruplets, or other type of multiplets of training peptide sequence vectors for which loss is computed.

The mining strategy may be, for example, an all-in strategy, a hard negative mining strategy, a semi-hard negative mining strategy, another type of strategy, or a combination thereof. In one or more embodiments, an all-in strategy refers to using all possible unique pairs of the training peptide sequence vectors in the batch of training peptide sequence vectors for evaluating loss. Hard negative mining and semi-hard negative mining are described in greater detail below in Section III.D.

In one or more embodiments, an all-in strategy is selected for a first portion of batches (e.g., batches up to batch number 10). A semi-hard negative mining strategy may be used for a second portion of the batches processed (e.g., batches between batch number 10 and batch number 40). A hard negative mining strategy may be used for a third portion of the batches processed (e.g., batches after batch number 40). In other embodiments, different combinations of mining strategies and/or different batch cutoffs may be used.

Step 510 includes forming an evaluation bundle from the batch of training peptide sequence vectors based on the identified mining strategy and the distance metrics. Step 510 may be performed by, for example, without limitation, loss evaluator 125 in FIG. 1. For example, step 510 may be performed by miner 135 of loss evaluator 125 in FIG. 1.

In one or more embodiments, the evaluation bundle is formed using each peptide sequence vector in the batch of training peptide sequence vectors. In other embodiments, the evaluation bundle is formed using a portion or subset of the batch of training peptide sequence vectors. The evaluation bundle includes groupings of training peptide sequence vectors (e.g., groupings formed by all or a subset of the training peptide sequence vectors in the batch). Each grouping includes at least two training peptide sequence vectors. For example, a grouping may be a pair, a triplet, a quadruplet, or some other multiplet or training peptide sequence vectors.

Step 510 may include forming groupings of training peptide sequence vectors from the batch of training peptide sequence vectors based on the identified mining strategy and the metric learning algorithm to be used for evaluating loss. The metric learning algorithm comprises at least one of a contrastive loss function, a triplet loss function, a quadruplet loss function, a circle loss function, a multi-class n-pair loss function, a lifted structure loss function, an angular loss function, a divergence loss function, or a constellation loss function. The metric learning algorithm may be, for example, metric learning algorithm 126 in FIG. 1.

When the metric learning algorithm takes the form of a triplet loss function, the groupings formed in the evaluation bundle in step 510 are triplets. The identified mining strategy determines how these triplets are formed.

Step 512 includes evaluating loss for the evaluation bundle using the metric learning algorithm. In one or more embodiments, step 512 may be performed by, for example, without limitation, loss evaluator 125 in FIG. 1. The loss may be evaluated in step 512 in various ways. For example, the loss may be evaluated per grouping (e.g., pair, triplet, etc.) of the evaluation bundle in which the various losses may be evaluated collectively. In some embodiments, the loss may be computed as a sum or average of a loss computed for each grouping in the evaluation bundle. In one or more embodiments, the loss may be evaluated such that the loss is low (1) when peptides that are presented by a same MHC allele are embedded (as training peptide sequence vectors) closer together in the n-dimensional space and (2) when peptides that are presented by different MHC alleles are embedded (as peptide sequence vectors) further away from each other in the n-dimensional space. Accordingly, the loss is high (1) when peptides that are presented by the same MHC allele are embedded further away from each other in the n-dimensional space and (2) when peptides that are presented by different MHC alleles are embedded closer together in the n-dimensional space. Examples of loss functions that may be used in step 512 are described in greater detail in Section III.C below.

Step 514 includes updating parameters of the machine learning model based on the loss. For example, step 514 may be performed to reduce or minimize the loss that is computed in step 512. The machine learning model parameters may also be referred to as weights in some instances. Updating the parameters in step 514 may include, for example, changing at least one parameter of the machine learning model. Thus, in some cases, a portion of the parameters may be changed and another portion unchanged, while in other cases, all of the parameters may be changed. In one or more embodiments, step 512 and step 514 are integrated together.

Step 518 includes determining whether unprocessed training peptide sequence vectors remain. If any unprocessed training peptide sequences remain, the process 500 returns to step 502 described above. Otherwise, if no unprocessed training peptide sequence vectors remain, the process 500 terminates, completing one epoch of processing. One epoch includes a complete pass of processing through the entire training peptide sequence data. Any number of epochs may be processed as part of process 500 to train the machine learning model. For example, process 500 may be repeated any number of times to train the machine learning model. In one or more embodiments, process 500 may be repeated until the loss evaluated in step 410 is within selected tolerances, until the machine learning model reaches convergence, or until the machine learning model reaches within selected tolerances of convergence.

Different implementations of process 500 may be used. In some embodiments, the parameters of the machine learning model may be updated after every epoch instead of after every batch of processing. For example, step 514 may be performed after one full epoch has been completed (e.g., after step 518).

Using a mining strategy such as, for example, hard negative mining or semi-hard negative mining in process 500 may reduce the overall computational resources and time that may be needed to evaluate loss and perform the training of the machine learning model. Further, using a mining strategy such as, for example, hard negative mining or semi-hard negative mining in process 500 may help the machine learning model move towards convergence more quickly and/or prevent a freeze in learning.

### III.C. Evaluating Loss: Overview of Loss Functions for Use in the Training of the Machine Learning Model

As described above with respect to the process 300 in FIG. 3, process 400 in FIG. 4, and process 500 in FIG. 5, a metric learning algorithm may be used to train a machine learning model to generate peptide sequence vectors for peptide sequences. The machine learning model may be, for example, model 124 in FIG. 1 or attention-based machine learning model 201 in FIG. 2. The metric learning algorithm, which may be, for example, metric learning algorithm 126 in FIG. 1, may include at least one of a contrastive loss function, a triplet loss function, a quadruplet loss function, a circle loss function, a multi-class n-pair loss function, a lifted structure loss function, an angular loss function, a divergence loss function, a constellation loss function, or some other type of distance-based loss function.

Various loss functions evaluate loss for groupings of peptide sequence vectors based on the classes to which these peptide sequence vectors belong. Here, class may be determined by the particular MHC allele that presents or is expected to present a given peptides, as identified by an allele identifier (e.g., a 4-digit, 6-digit, or 8-digit allele identifier). For example, two peptide sequence vectors that represent peptide sequences (and thereby, peptides) that are presented by a same MHC allele (e.g., MHC alleles with the same allele identifier) belong to a same class. Two peptide sequence vectors that represent peptide sequences (and thereby, peptides) that are presented by two different MHC alleles (e.g., MHC alleles with different allele identifiers) belong to different classes.

### III.C.1. Contrastive Loss

A contrastive loss function focuses on evaluating one pair of peptide sequence vectors at a time. If the two peptide sequence vectors belong to a same class (i.e., represent peptides that are presented by a same MHC allele), the loss is higher when the peptide sequence vectors are further apart and lower when the peptide sequence vectors are closer together. If the two peptide sequence vectors belong to different classes (i.e., represent peptides that are presented by different MHC alleles), the loss is higher when the peptide sequence vectors are closer together and lower when the peptide sequence vectors are further apart.

In one or more embodiments, one example of a contrastive loss function that can be used to evaluate a single pair of peptide sequence vectors is defined as follows:

$$( 1 - Y ) \frac{1}{2} {( {D}_{W} )}^{2} + ( Y ) \frac{1}{2} {\{ max ( 0 , m - {D}_{W} ) \}}^{2} \qquad ( 2 )$$

where:
Y=0 for a pair of peptide sequence vectors belonging to different classes;
Y=1 for a pair of peptide sequence vectors belonging to a same class;
D_{W} is the distance (e.g., Euclidean distance) between the two peptide sequence vectors in a pair; and
m is the margin.

In one or more embodiments, the margin is a constant set so that the loss function penalizes the model if the distance between peptide sequence vectors belonging to different classes is less than m. However, if this distance is less than or equal to m, the loss function is set to zero. This ensures that the peptide sequence vectors of different classes are not forced further apart than is necessary.

Referring back to FIG. 5, in some embodiments, evaluating loss in step 512 in FIG. 5 may be performed by evaluating the loss for each pair of peptide sequence vectors in the evaluation bundle. In other embodiments, evaluating loss in step 512 may be performed by summing, averaging, or otherwise combining or integrating the losses computed for each pair of peptide sequence vectors in the evaluation bundle.

### III.C.2. Triplet Loss Function

A triplet loss function evaluates loss based on triplets that each include an anchor, a positive example, and a negative example. The anchor peptide sequence vector is the peptide sequence vector against which the positive and negative are defined. For example, the positive example may be a peptide sequence vector of the same class as the anchor. In other words, the positive example and the anchor may represent peptide sequences that are presented by a same MHC allele. The negative example may be a peptide sequence vector of a different class than the anchor. In other words, the negative example and the anchor may represent peptide sequences presented by different MHC alleles. With the triplet loss function, the machine learning model is trained to increase (e.g., maximize) the distance between the anchor and the negative example and reduce (e.g., minimize) the distance between the anchor and the positive example.

In one or more embodiments, one example of a triplet loss function is defined as follows:

$$L ( {r}_{a} , {r}_{p} , {r}_{n} ) = max ( 0 , m + d ( {r}_{a} , {r}_{p} ) - d ( r , {r}_{n} ) ) \qquad ( 3 )$$

where rₐ is the anchor representation (e.g., anchor peptide sequence vector); rₚ is the positive representation (e.g., positive peptide sequence vector belonging to a same class as the anchor peptide sequence vector); rₙ is the negative representation (e.g., peptide sequence vector belonging to a different class than the anchor peptide sequence vector); d() is the distance function; and m is the margin. The margin may be a constant set based on the objective that the distance between the anchor and the negative should be greater than the margin m.

Referring back to FIG. 5, in some embodiments, evaluating loss in step 512 in FIG. 5 may be performed by evaluating the loss for each triplet in the evaluation bundle. In other embodiments, evaluating loss in step 512 may be performed by summing, averaging, or otherwise combining or integrating the losses computed for each triplet in the evaluation bundle.

### III.C.3. Other Examples of Loss Functions

Any number of other loss functions may be used by the metric learning algorithm. Regardless of the loss function selected, the metric learning algorithm trains the machine learning algorithm to increase the distance between peptide sequence vectors belonging to different classes and reduce the distance between peptide sequence vectors belonging to the same class.

A quadruplet loss function builds upon the concepts involved in the triplet loss function. With a quadruplet loss function, loss is evaluated for a quadruplet that includes an anchor peptide sequence vector, a positive peptide sequence vector (belonging to a same class as the anchor), and two negative peptide sequence vectors (belonging to one or more different classes with respect to the anchor).

In one or more embodiments, one example of a quadruplet loss function is defined as follows:

$$L ( {r}_{a} , {r}_{p} , {r}_{n 1} , {r}_{n 2} ) = max ( 0 , m + d ( {r}_{a} , {r}_{p} ) - d ( {r}_{a} , {r}_{n 1} ) ) + max ( 0 , m + d ( {r}_{a} , {r}_{p} ) - d ( {r}_{n 1} , {r}_{n 2} ) ) \qquad ( 4 )$$

where rₐ is the anchor representation (e.g., anchor peptide sequence vector); rₚ is the positive representation (e.g., positive peptide sequence vector belonging to a same class as the anchor peptide sequence vector); rₙ₁ is a first negative representation (e.g., a first negative peptide sequence vector belonging to a different class than the anchor peptide sequence vector); rₙ₂ is a second negative representation (e.g., a second negative peptide sequence vector belonging to a different class than the anchor peptide sequence vector, the positive peptide sequence vector, and the first negative sequence vector); d() is the distance function; and m is the margin. The margin may be a constant set based on the objective that the distance between the anchor and the negative should be greater than the margin m.

A lifted structure loss function also builds upon the concepts involved in the triplet loss function and quadruplet loss function. The lifted structure loss function uses multiple negative examples in a mini batch. The multiple negative examples include negatives of not just the anchor but also of the positive. Thus, the lifted structure loss function analyzes the distances between all possible pairs within a mini-batch. For example, a mini-batch may include examples: x1, x2, x3, x4, x5, and x6, where x1 and x2 belong to the same class, x3 and x4 belong to the same class, and x5 and x6 belong to the same class. All other pairings may be of different classes such that:
x1 belongs to a different class than x3, x4, x5, and x6;
x2 belongs to a different class than x3, x4, x5, and x6;
x3 belongs to a different class than x1, x2, x5, and x6;
x4 belongs to a different class than x1, x2, x5, and x6;
x5 belongs to a different class than x1, x2, x3, and x4; and
x6 belongs to a different class than x1, x2, x3, and x4.

In one or more embodiments, one example of the lifted structure loss function is as follows:

$${L}_{i , j} = log ( {\sum }_{( i , k ) ∈ N}^{} {e}^{α - {D}_{i , k}} + {\sum }_{( i , l ) ∈ N}^{} {e}^{α - {D}_{j , l}} ) + {D}_{i , j} \qquad ( 5 )$$

$$L = \frac{1}{2 \vert P \vert} {\sum }_{( i , j ) ∈ P}^{} {max ( 0 , {L}_{i , j} )}^{2} \qquad ( 6 )$$

where Dᵢⱼ=∥f(xᵢ)−f(xᵢ)∥₂; P is all positive pairs in the mini-batch; N is all negative pairs in the mini-batch;

A multi-class n-pair loss randomly selects a negative example in each class for a grouping. For example, a grouping may be formed using an anchor peptide sequence vector, a positive peptide sequence vector, and a plurality of negative peptide sequence vectors. The anchor peptide sequence vector may represent a peptide presented by a particular MHC allele (e.g., allele A) and the plurality of negative peptide sequence vectors may include a randomly selected peptide sequence vector for every different MHC allele (e.g., a peptide sequence vector corresponding to every non-A allele).

A circle loss function seeks to provide different levels of optimization for different types of triplets. For example, a first triplet T may include an anchor A, a positive P, and a negative N. A second triplet T′ may include the anchor A, a positive P′ and a negative N′. If P′ is much further away than P, the circle loss function will place more emphasis on reducing the distance between A and P′ for the second triplet T′ and more emphasis on increasing the distance between A and N for the first triplet T. Thus, different penalties may be associated with different triplets based on the distances between the anchor and positive and the anchor and negative. These distances may be weighted independently and can be optimized at different paces.

An angular loss function looks at cosine distance with respect to triplets. A triplet of peptide sequence vectors may form a triangle. The angular loss function evaluates the angle between a first edge formed by the anchor and the positive and a second edge formed by the anchor and the negative. The angular loss function seeks to push the negative away from both the anchor and the positive and to pull the anchor and the positive closer together. The angular loss function may be used with other loss functions (e.g., triplet loss, multi-class n-pair loss, etc.) to improve overall performance.

A divergence loss function may be used for regularization when an ensemble of learners (e.g., models and/or loss functions) is used. For example, for training peptide sequences that are sent as input through multiple models (e.g., each implemented using model 124 in FIG. 1 or attention-based machine learning model 201 in FIG. 2), the divergence loss function causes the different models to embed the training peptide sequences with a focus on different features such that diverse embeddings are generated. In this manner, the models may have diverse embedding spaces but all may still satisfy the constraint of embedding similar peptide sequences closer together and dissimilar peptide sequences further apart.

A constellation loss function merges the concepts of the triplet loss function and the multi-class n-pair loss function. With the constellation loss function, the distances between different class combinations are simultaneously learned. For example, similar to the multi-class n-pair loss function, a grouping may include the anchor, a positive, and a negative from each possible class. Constellation loss evaluates the distance between the anchor and positive, the distances between the anchor and each of the negatives, and the distances between each negative and every other negative.

The above-described loss functions are but some of the different types of loss functions that may be included in a metric learning algorithm such as, for example, metric learning algorithm 126 in FIG. 1. Other types of loss functions may also be utilized. Further, a mixed loss function that includes two or more of the above-described and/or other loss functions may be utilized.

### III.D. Mining (Sampling) Strategies

### III.D.1. Mining Strategies, Generally

Different types of mining strategies may be used when determining how to evaluate loss for a machine learning model. A mining strategy, which may also be referred to as a sampling strategy, is a strategy for selecting groupings of peptide sequence vectors (or embeddings) for evaluating loss. A grouping may include at least two peptide sequence vectors. For example, a grouping may be a pair, triplet, quadruplet, or other multiplet of peptide sequence vectors.

Generally, a miner, such as miner 135 in FIG. 1, may take the form of a subset batch miner, a tuple miner, or another type of miner. A subset batch miner may take a batch of training peptide sequence vectors (e.g., the batch of training peptide sequence vectors generated in step 504 in FIG. 5) and return a subset that is to be used by a tuple miner or by a loss function (e.g., of the metric learning algorithm). A tuple miner may take a batch of training peptide sequence vectors and return a certain number of tuples (e.g., pairs, triplets, quadruplets, etc.) to be used for computing the loss.

The peptide sequence vectors may be mined based on their class, which, as used herein, refers to the particular MHC allele expected (or predicted) to present the peptide corresponding to a peptide sequence vector. The MHC allele may be identified via an allele identifier (e.g., a 4-digit, 6-digit, or 8-digit allele identifier). A tuple (e.g., pair, triplet, quadruplet, etc.) may include an anchor peptide sequence vector (or simply, an anchor) and at least one of a positive peptide sequence vector (or simply, a positive) or a negative peptide sequence vector (or simply, a negative). A positive is a peptide sequence vector that belongs to a same class as the anchor. A negative is a peptide sequence vector that belongs to a different class than the anchor or than the positive.

Two examples of tuple miners include a pair miner and a triplet miner. In one or more embodiments, a pair miner may be used with, for example, a contrastive loss function. The pair miner may receive M peptide sequence vectors (embeddings) and output T tuples of size 4, with each tuple including an anchor-positive pair and an anchor-negative pair. In other embodiments, a pair miner may receive M peptide sequence vectors (embeddings) and output P anchor-positive pairs and P anchor-negative pairs for processing via the loss function. Without a pair miner, a contrastive loss function may evaluate, by default, all possible pairs in the training batch.

In one or more embodiments, a triplet miner may be used with, for example, a triplet loss function as well as other types of loss functions that evaluate triplets. The triplet miner may receive M peptide sequence vectors (embeddings) and output T triplets, with each triplet including an anchor, a positive, and a negative. Without a triplet miner, a contrastive loss function may use, by default, all possible triplets in the training batch.

But not all positives and negatives may be considered of equal difficulty or interest for training purposes. Some positives may be harder or more challenging in the sense that they are less similar (e.g., are further apart distance-wise) to the anchor than other positives. Some negatives may be harder or more challenging in the sense that they are more similar (e.g., are closer together distance-wise) to the anchor than other negatives.

In one or more embodiments, the positive and/or negative selected for a tuple is selected based on a selected positive strategy and/or a selected negative strategy, respectively. The selected positive strategy may include using hard positives, semi-hard positives, easy positives, and/or all positives. For example, a hard positive strategy may include returning the hardest positive per anchor or, in other words, the positive that is most dissimilar to (furthest away from) the anchor. An easy positive strategy may include returning the easiest positive per anchor or, in other words, the positive that is most similar to (closest to) the anchor. A semi-hard positive strategy, which is typically used by a triplet miner, may include returning a semi-hard positive per anchor. The semi-hard positive may be, for example, the hardest positive that is still easier than the selected negative or, in other words, the positive that is most dissimilar from (furthest away from) the anchor but still more similar (closer than) than the selected negative. In some cases, the selected positive strategy used in one or more batches may be different from the positive strategy used in other batches.

The selected negative strategy may include using hard negatives, semi-hard negatives, easy negatives, and/or all negatives. For example, a hard negative strategy may include returning the hardest negative per anchor or, in other words, the negative that is most similar to (closest to) the anchor. An easy negative strategy may include returning the easiest negative per anchor or, in other words, the negative that is most dissimilar to (furthest away from) the anchor. A semi-hard negative strategy, which is typically used by a triplet miner, may include returning a semi-hard negative per anchor. The semi-hard negative may be, for example, the hardest negative that is still easier than the selected positive or, in other words, the negative that is most similar to (closest to) the anchor but still more dissimilar than (further away than) than the selected positive. In some cases, the selected negative strategy used in one or more batches may be different from the negative strategy used in other batches.

When a hard negative strategy is used, the overall mining strategy may be referred to as hard negative mining (or online hard negative mining) regardless of the type of positive strategy used. When a semi-hard negative strategy is used, the overall mining strategy may be referred to as semi-hard negative mining regardless of the type of positive strategy used.

In other embodiments, one or more other types of miners may be used to prioritize certain pairs or triplets of peptide sequence vectors based on loss for training. Other types of miners include, but are not limited to, an angular miner, a base miner, a base tuple miner, a base subset batch miner, a batch easy hard miner, a batch hard miner, a distance weighted miner, a miner for embeddings already packaged as triplets, a hard-aware deeply cascaded embedding miner, a maximum loss miner, a multi-similarity miner, a pair margin miner, a triplet margin miner, and a uniform histogram miner. In some cases, a user may create a customized miner.

III.D.2. Hard Negative Mining and Semi-Hard Negative Mining for Use with Triplet Loss Function

With the triplet loss function, triplets that include an anchor, a positive, and a negative are formed. Hard negative mining semi-hard negative mining for the triplet loss function may be determined relative to the positive selected for a triplet.

Hard negative mining and semi-hard negative mining are both performed to exclude the evaluation of triplets that include easy negatives. An easy negative may be defined relative to the distance between the anchor and the selected positive and a margin m. For example, an easy negative satisfies the following constraint:

$$d ( {r}_{a} , {r}_{n} ) > d ( {r}_{a} , {r}_{p} ) + m . \qquad ( 7 )$$

With hard negative mining, hard triplets are formed using hard negatives. Hard negatives may be those peptide sequence vectors that are closer to the anchor than the selected positive. In other words, the distance between the anchor and the negative is less than the distance between the anchor and the positive. Thus, a hard triplet satisfies the following constraint:

$$d ( a , n ) < d ( a , p ) . \qquad ( 8 )$$

With hard negative mining, each hard triplet is formed using the hardest negative for the corresponding anchor of that triplet. In other words, the negative that is closest to the anchor is selected.

For semi-hard negative mining, each triplet is formed using an anchor, a positive, and a semi-hard negative. The semi-hard negative that is chosen may be further away from the anchor than the selected positive but may be closer to the anchor than any easy negatives. Thus, a semi-hard triplet satisfies the constraint:

$$d ( a , p ) < d ( a , n ) < d ( a , p ) + m . \qquad ( 9 )$$

In one or more embodiments, with semi-hard negative mining, the most challenging semi-hard negative may be selected. For example, the negative that is closest to the anchor but that satisfies the above constraint may be selected. In other examples, the negative may be randomly selected from the possible semi-hard negatives.

Using hard negative mining, semi-hard negative mining, or both helps prevent or reduce the possibility of collapse during training in which the training freezes or stalls. Such mining strategies allow faster convergence to a trained machine learning model with improved accuracy. For example, given a batch of randomly selected peptide sequence vectors, there may be numerous anchor-negative pairs that can be chosen. Randomly choosing which anchor-negative pairs to use for evaluating loss may result in pairs that are too easy such that the machine learning model does not sufficiently learn from these pairs, resulting in poor performance.

### III.E. Generating Peptide Sequence Data

### III.E.1. Example Processing of Samples

The embodiments described herein provide a machine learning model that can be used to generate peptide sequence vectors for peptide sequences that can be used in the selection of a diverse group of candidate peptides for a peptide therapeutic. These peptide sequences may be identified from one or more samples (e.g., set of samples 118 in FIG. 1) from one or more subjects.

In one or more embodiments, the peptide sequences that are of interest for the development of a peptide vaccine (e.g., a neoantigen vaccine) are disease-specific peptide sequences. However, some of the peptide sequences identified in a disease sample may be non-disease peptide sequences that correspond to non-disease peptides. To identify whether each sequence that is detected as a result of sequencing the disease-specific sample is a disease-specific peptide sequence (e.g., disease-specific nucleic acid sequence and/or disease-specific amino-acid sequence), it may be determined whether the sequence is also identified in a reference sequence data set. The reference sequence data set can include a set of reference peptide sequences for which it is known, inferred or assumed that the peptide sequence is not indicative or characteristic of a disease (e.g., any disease or a given disease). The reference sequence data set may, for example, include peptide sequences identified by sequencing one or more reference sample sequences collected from a same subject from which the disease-specific sample was collected, sequencing one or more reference sample sequences collected from one or more other subjects not diagnosed with any disease or a disease corresponding to the disease-specific sample and/or sequencing one or more cell lines not associated with the specific disease. In some instances, the reference sequence data set may include peptide sequences collected from one or more reference data repositories. A peptide sequence that is detected in association with the disease-specific sample but that is not detected (or detected at a frequency below a pre-defined threshold) in a reference sequence data set can be classified as a variant-coding peptide sequence (e.g., generally or for a subject from which the disease-specific sample was collected).

In some instances, multiple variant-coding peptide sequences may be identified (e.g., each having been detected in the disease sample but not being represented in the reference-sample sequences), and a representation of each of the multiple variant-coding sequences can be processed (e.g., individually, sequentially and/or in parallel) using a machine learning model disclosed herein (e.g., an attention-based machine learning model such as attention-based machine learning model 201 in FIG. 2).

The disease sample can include, for example, tissue (e.g., a solid tumor), blood and/or a collection of cells (e.g., cancer cells, which may have been collected using fine need aspiration or laparoscopy). The disease sample may include cancerous cells collected from a subject that has been diagnosed with and/or that has, for example, lung cancer, melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, and T cell lymphocytic leukemia, non-small cell lung cancer, or small cell lung cancer.

In some instances, an initial sample is separated into a disease sample and another remainder sample (e.g., which may be discarded or used as a reference sample). The reference sample can include a matched disease-free sample. Each of the disease sample and the reference sample may be collected from a same subject and/or may include or may be of a same or similar sample type (e.g., tissue type). In some instances, the disease sample is collected from a first subject (e.g., who has been diagnosed with a medical condition or disease), and the reference sample is collected from a different second subject (e.g., who has not been diagnosed with the medical condition or disease). In some instances, the reference-sample peptide sequences are retrieved from a database of known genes associated with an organism.

With respect to any type of sequencing (e.g., to identify sequences in a sample, peptides bound to an MHC molecule, HLA typing, etc.), a result may identify one or more nucleic acid sequences or one or more amino-acid sequences. When nucleic acid sequences are identified and an attention-based model (or other processing) is configured to process amino-acid sequences, a technique (e.g., lookup table) may be used to convert individual codons within the nucleic acid sequences into individual amino acids.

### III.E.2. Example of Identification of Peptide Sequence Data: Mutant Peptide Sequences

When developing a peptide therapeutic, mutant peptides are selected to trigger the desired immunological response. The mutant peptides may be disease-specific peptides and may be, for example, specific to the individual subject who has the disease. Accordingly, peptide sequence data 116 in FIG. 1 may be mutant peptide sequence data. In particular, peptide sequences 122 for peptides 120 in FIG. 1 may be mutant peptide sequences for mutant peptides that are detected in a disease sample collected from a subject but that are not observed in one or more non-disease samples (e.g., from the subject or another subject).

A variety of methods may be available for identifying mutant peptide sequences and thereby, their corresponding mutant peptides with a given subject. Mutations can be present in the genome, transcription, proteome or exome of diseased cells of a subject but not in a non-diseased sample, for example, a non-diseased sample from the subject or from another subject. Mutations include, but are not limited to, (1) non-synonymous mutations leading to different amino acids in the protein; (2) read-through mutations in which a stop codon is modified or deleted, leading to translation of a longer protein with a novel tumor-specific sequence at the C-terminus; (3) splice site mutations that lead to the inclusion of an intron in the mature mRNA and thus a unique tumor-specific protein sequence; (4) chromosomal rearrangements that give rise to a chimeric protein with tumor-specific sequences at the junction of 2 proteins (i.e., gene fusion); (5) frameshift insertions or deletions that lead to a new open reading frame with a novel tumor-specific protein sequence. Mutations can also include one or more of non-frameshift insertion/deletion (indel), missense or nonsense substitution, splice site alteration, genomic rearrangement or gene fusion, or any genomic or expression alteration giving rise to a neoORF.

Peptides with mutations or mutated polypeptides arising from, for example, splice-site, frameshift, readthrough, or gene fusion mutations in diseased cells can be identified by sequencing DNA, RNA or protein in the diseased sample and comparing the obtained sequences with sequences from a non-diseased sample.

In some embodiments, whole genome sequencing (WGS) or whole exome sequencing (WES) data from a disease sample and a non-diseased sample can be obtained and compared. Following the alignment of non-diseased sample and diseased sample reads to the human reference genome, somatic variants, which include single nucleotide variants (SNV), gene fusions and insertion or deletion variants (indels), can be detected using variant-calling algorithms. One or more variant callers can be used to detect different somatic variant types (i.e., SNV, gene fusions, or indels).

In some examples, the mutant peptides are identified based on the transcriptome sequences in the disease sample from the individual. For example, whole or partial transcriptome sequences (for example by methods such as RNA-Seq) can be obtained from a diseased tissue of the individual and subjected to sequencing analysis. The sequences obtained from the diseased tissue sample can then be compared to those obtained from a reference sample. Optionally, the diseased tissue sample is subjected to whole-transcriptome RNA-Seq. Optionally, the transcriptome sequences are “enriched” for specific sequences prior to the comparison to a reference sample. For example, specific probes can be designed to enrich certain desired sequences (for example disease-specific sequences) before being subjected to sequencing analysis.

In some embodiments, transcriptomic sequencing techniques include, but are not limited to, RNA poly(A) libraries, microarray analysis, parallel sequencing, massively parallel sequencing, PCR, and RNA-Seq. RNA-Seq is a high-throughput technique for sequencing part of, or substantially all of, the transcriptome. In short, an isolated population of transcriptomic sequences is converted to a library of cDNA fragments with adaptors attached to one or both ends. With or without amplification, each cDNA molecule is then analyzed to obtain short stretches of sequence information, typically 30-400 base pairs. These fragments of sequence information are then aligned to a reference genome, reference transcripts, or assembled de novo to reveal the structure of transcripts (i.e., transcription boundaries) and/or the level of expression.

Once obtained, the peptide sequences in the disease sample can be compared to the corresponding peptide sequences in a reference sample. The sequence comparison can be conducted at the nucleic acid level, by aligning the nucleic acid sequences in the disease tissue with the corresponding sequences in a reference sample. Genetic sequence variations that lead to one or more changes in the encoded amino acids are then identified. Alternatively, the sequence comparison can be conducted at the amino acid level, that is, the nucleic acid sequences are first converted into amino acid sequences in silico before the comparison is carried out. Either the amino-acid-based approach or the nucleic acid-based approach can be used to identify one or more mutations (e.g., one or more point mutations) in the peptide. With regard to nucleic acid-based approaches, the discovered variants can be used to identify one or more nucleic acid sequences (e.g., DNA sequences, RNA sequences or mRNA sequences) that would give rise to a given observable mutant protein (e.g., via a look-up table that associated individual peptide mutations with multiple codon variants).

In some embodiments, comparison of a peptide sequence from the disease sample to those of a reference sample can be completed by techniques known in the art, such as manual alignment, FAST-All (FASTA), and Basic Local Alignment Search Tool (BLAST). In some embodiments, comparison of a sequence from a disease sample to those of a reference sample can be completed using a short read aligner, for example GSNAP, BWA, and STAR.

In some embodiments, the reference sample is a matched, disease-free sample. As used herein, a “matched,” disease-free tissue sample is one that is selected from the same or similar sample, for example, a sample from the same or similar tissue type as the disease sample. In some embodiments, a matched, disease-free tissue and a disease tissue may originate from the same individual. The reference sample described herein in some embodiments is a disease-free sample from the same individual. In some embodiments, the reference sample is a disease-free sample from a different individual (for example an individual not having the disease). In some embodiments, the reference sample is obtained from a population of different individuals. In some embodiments, the reference sample is a database of known genes associated with an organism. In some embodiments, a reference sample may be from a cell line. In some embodiments, a reference sample may be a combination of known genes associated with an organism and genomic information from a matched disease-free sample. In some embodiments, a variant-coding sequence may comprise a point mutation in the amino acid sequence. In some embodiments, the variant-coding sequence may comprise an amino acid deletion or insertion.

In some embodiments, the set of variant-coding sequences are first identified based on genomic and/or nucleic acid sequences. This initial set is then further filtered to obtain a narrower set of expression variant-coding sequences based on the presence of the variant-coding sequences in a transcriptome sequencing database (and is thus deemed “expressed”). In some embodiments, the set of variant-coding sequences are reduced by at least about 10, 20, 30, 40, 50, or more times by filtering through a transcriptome sequencing database.

Alternatively, protein mass spectrometry can be used to identify or validate the presence of mutant peptides, for example, mutant bound to MHC proteins on tumor cells. Peptides can be acid-eluted from diseased cell, for example, tumor cells or from HLA molecules that are immunoprecipitated from the tumor, and then identified using mass spectrometry.

A mutant peptide can have, for example, 5 or more, 8 or more, 11 or more, 15 or more, 20 or more, 40 or more, 80 or more, 100 or more, 120 or fewer, 100 or fewer, 80 or fewer, 60 or fewer, 50 or fewer, 40 or fewer, 30 or fewer, 25 or fewer, 20 or fewer, 18 or fewer, 15 or fewer, or 13 or fewer amino acids.

Tumor-specific T-cell receptor sequences can also be identified, for example, by single cell T-cell receptor sequencing. High-throughput sequencing of T cell repertoires can also or alternatively be performed to identify tumor-specific signatures for a particular disease.

### III.E.3. Example of Identification of Training Peptide Sequence Data

The training of a machine learning model, such as model 124 in FIG. 1 and/or attention-based machine learning model 201 in FIG. 2, may be performed using training peptide sequence data, such as training peptide sequence data 130 in FIG. 1. This training peptide sequence data may be generated in different ways, including those described above in Sections III.E.1 and III.E.2.

In one or more embodiments, the peptide sequence data described above in Sections III.E.1 and III.E.2 may form at least a portion of the training peptide sequence data. In other embodiments, the training peptide sequence data can be generated using data collected from multiple other samples (e.g., potentially being associated with one or more other subjects). Each of the multiple other samples can include, for example, tissue (e.g., a biopsy), single cell, multiple cells, fragments of cells or an aliquot of body fluid. In some instances, the multiple other samples are collected from a different type of subject as compared to a subject associated with input data to be processed by the trained model. For example, the machine learning model may be trained using training peptide sequence data collected by processing samples from one or more cell lines, and the trained machine learning model may be used to process input data determined by processing one or more samples from a human subject.

### IV. Pharmaceutically Acceptable Composition and Manufacture

A pharmaceutically acceptable composition may be developed and/or manufactured based on, for example, the diverse group of candidate peptides (e.g., diverse group of candidate peptides 110 in FIG. 1) identified using a machine learning model (e.g., model 124 in FIG. 1., attention-based machine learning model 201 in FIG. 2) described herein. For example, the peptide sequence vectors generated by the machine learning model may be used to select the diverse group of candidate peptides. Treatment peptides (e.g., treatment peptides 144 in FIG. 1) may be formed from the diverse group of candidate peptides for development of the pharmaceutically acceptable composition. The treatment peptides include two or more candidate peptides from the diverse group of candidate peptides. The pharmaceutically acceptable composition may be a peptide therapeutic (e.g., peptide therapeutic 108 in FIG. 1). For example, the pharmaceutically acceptable composition may be a peptide vaccine (e.g., a tumor vaccine). The two or more candidate peptides selected from the diverse group of candidate peptides include at least two dissimilar peptides (e.g., having dissimilar binding motifs).

The treatment peptides may be, for example, mutant peptides, which may be identified by their corresponding variant-coding sequences. The composition may include the mutant peptides, precursors to the mutant peptides, polypeptide sequences corresponding to the mutant peptides, RNA (e.g., mRNA) corresponding to the mutant peptides, DNA corresponding to the mutant peptides, cells that include the mutant peptides and/or one or more nucleic acids encoding such peptides, plasmids corresponding to the mutant peptides, and/or vectors corresponding to the mutant peptides.

In one or more embodiments, a pharmaceutically acceptable composition may be developed and/or manufactured using the selected variant-coding sequences for the mutant peptides. The composition may include mutant peptides corresponding to a single selected variant-coding sequence. The composition may include mutant peptides and/or mutant-peptide precursors corresponding to multiple selected variant-coding sequences.

Each of one, more or all of the mutant peptides in the composition can have, for example, a length of about 7 to about 40 amino acids (e.g., about any of 7, 8, 9, 10, 11, 12, 13, 14, 15, 17, 20, 22, 25, 30, 35, 40, 45, 50, 60 or 70 amino acids in length). In some embodiments, a length of each of one, more or all of the mutant peptides in the composition are within a predefined range (e.g., 8 to 11 amino acids, 8 to 12 amino acids or 8 to 15 amino acids). In some embodiments, each of one, more or all of the mutant peptides in the composition is about 8 to 10 amino acids in length. Each of one, more or all of the mutant peptides in the compositions may be in its isolated form. Each of one, more of all of the mutant peptides in the composition may be a “long peptide” produced by adding one or more peptides to an end (or to each end) of the mutant peptide. Each of one, more or all of the mutant peptides in the composition may be tagged, may be a fusion protein, and/or may be a hybrid molecule.

A pharmaceutically acceptable composition may be developed and/or manufactured to include or by using one or more nucleic acids that encode-for each of one, more or all of the selected variant-coding sequences—a peptide that includes or is composed by amino acids as identified in a variant-coding sequence. The nucleic acid(s) can include DNA, RNA and/or mRNA. Given that any of multiple codons can encode a given amino acid, the codons may be selected to, for example, optimize or promote expression in a given type of organism. Such selection may be based on a frequency that each of multiple potential codons are used by the given type of organism, the translational efficiency of each of multiple potential codons in the given type of organism, and/or the given type of organism's degree of bias towards each of the multiple potential codons.

In some instances, the pharmaceutically acceptable composition may include one or more nucleic acids encoding the mutant peptides or precursors of the mutant peptides described above. For example, the pharmaceutically acceptable composition may be a nucleic acid vaccine. The nucleic acid vaccine may be, for example, an individualized vaccine that is specific to (e.g., and potentially developed for) a particular subject. The nucleic acid vaccine may include a nucleic acid that encodes the mutant peptides or precursors of the mutant peptides. The nucleic acid vaccine may include sequences flanking the sequence coding the mutant peptides (or precursors thereof). In some instances, the nucleic acid vaccine includes epitopes corresponding to the selected variant-coding sequences. The nucleic acid vaccine may be a DNA-based vaccine, an RNA-based vaccine, an mRNA-based vaccine, or a modified mRNA vaccine (e.g., including modified mRNA that is protected from degradation using protamine, mRNA containing modified 5′-cap structure, or mRNA containing modified nucleotides). In some embodiments, the RNA-based vaccine comprises single-stranded mRNA.

A nucleic acid vaccine may include an individualized neoantigen specific therapy manufactured for a particular subject to be used as part of next-generation immunotherapy. The individualized vaccine may have been designed by first detecting mutant peptides in a sample of the particular subject and then selecting dissimilar peptides from a group of peptides that have been identified as having the highest presentation potential. For each selected mutant peptide, a synthetic mRNA sequence can be identified that codes for the mutant peptide. An mRNA vaccine may include mRNA (that encodes part or all of a mutant peptide) complexed with lipids to form an mRNA-lipoplex. Administration of a vaccine that includes the mRNA-lipoplex can result in the mRNA stimulating TLR7 and TLR8, triggering T-cell activation by dendritic cells. Further, the administration can result in translation of mRNA into a mutant peptide, which can then bind to and be presented by MHC molecules and induce T-cell response.

In one or more embodiments, the composition may include multiple polynucleotide constructs (e.g., DNA constructs or RNA constructs). A polynucleotide construct is an artificially constructed segment of nucleic acid which may be transplanted into a target tissue or cell. The polynucleotide construct comprises a DNA or RNA (e.g., mRNA) insert, which contains the nucleotide sequence encoding the mutant peptides. In order to increase antigen presentation (e.g., presentation of the mutant peptides by the MHC molecules), the polynucleotide construct may further comprise a modification developed for improved antigen presentation, and thus improved immunogenicity to the mutant peptides. In some instances, the modification is incorporation of a transmembrane region and a cytoplasmic region of a chain of the MHC molecule into the polynucleotide construct as described in International Publication WO2005038030A1, which is incorporated herein by reference in its entirety for all purposes.

To provide an RNA insert with increased stability and translation efficiency, the polynucleotide construct may further comprise a modification developed for improved stability and translation, and thus improved immunogenicity to the mutant peptides. In some instances, the modification is incorporation of a nucleic acid sequence with at least two copies of a 3′ untranslated region (UTR) of a human beta-globin gene into a polynucleotide construct as described in International Publication WO2007036366A2, which is incorporated herein by reference in its entirety for all purposes. In other instances, the modification is incorporation of a nucleic acid sequence that codes for a 3′ UTR such as F1 3′ UTR described in International Publication WO2017060314A3, which is incorporated herein by reference in its entirety for all purposes.

To provide an RNA insert with increased stability and expression, the polynucleotide construct may further comprise a modification developed for improved stability and expression, and thus improved immunogenicity to the selected mutant peptides. In some instances, the modification is incorporation of a cap on an end of the RNA such as a 5′-cap structure. The cap structure may be the D1 diastereomer of beta-S-ARCA as described in International Publication WO2011015347A1, which is incorporated herein by reference in its entirety for all purposes.

In order to deliver the polynucleotide construct with high selectivity to antigen presenting cells, the composition may further include cationic liposomes or a lipoplex for improved uptake of the polynucleotide construct, and thus improved immunogenicity to the selected mutant peptides. In some instances, the composition includes nanoparticles comprising the polynucleotide construct. The nanoparticles may be lipoplexes comprising one or more lipids such as DOTMA and DOPE as described in International Publication WO2013143683A1, which is incorporated herein by reference in its entirety for all purposes.

The composition may include substantially pure mutant peptides, substantially pure precursors thereof, and/or substantially pure nucleic acids encoding the mutant peptides or precursors thereof. The composition may include one or more suitable vectors and/or one or more delivery systems to contain the mutant peptides, precursors thereof, and/or nucleic acids encoding the mutant peptides or precursors thereof. Suitable vectors and delivery systems include viral, such as systems based on adenovirus, vaccinia virus, retroviruses, herpes virus, adeno-associated virus or hybrids containing elements of more than one virus. Non-viral delivery systems include cationic lipids and cationic polymers (e.g., cationic liposomes). In some embodiments, physical delivery, such as with a ‘gene-gun’ may be used.

The composition may include cells comprising the mutant peptides and/or nucleic acid(s) encoding the mutant peptides described above. The composition may further comprise one or more suitable vectors and/or one or more delivery systems for the mutant peptides and/or nucleic acid(s) encoding the mutant peptides. In some instances, the cells comprising the mutant peptides and/or nucleic acid(s) encoding the mutant peptides are non-human cells, for example, bacterial cells, protozoan cells, fungal cells, or non-human animal cells. In some instances, the cells comprising the mutant peptide and/or nucleic acids encoding the mutant peptide are human cells. In some instances, the human cells are immune cells. In some instances, the immune cells are antigen-presenting cells (APCs). In some instances, the APCs are professional APCs, such as macrophages, monocyte, dendritic cells, B cells, and microglia. In other instances, the professional APCs are macrophages or dendritic cells. In some instances, the APCs comprising the mutant peptides and/or nucleic acid sequence(s) encoding the mutant peptides are used as a cellular vaccine, thereby inducing a CD4+ or a CD8+ immune response. In other instances, the composition used as a cellular vaccine includes mutant peptide-specific T cells primed by APCs comprising the mutant peptides and/or nucleic acid sequence(s) encoding the mutant peptides.

The composition may include a pharmaceutically acceptable adjuvant and/or pharmaceutically acceptable excipient. Adjuvants refer to any substance for which admixture into a composition modifies an immune response to a mutant peptide. Adjuvants may be conjugated using, for example, an immune stimulation agent. Excipients can increase the molecular weight of a particular mutant peptide to increase activity or immunogenicity, confer stability, increase biological activity, and/or increase serum half-life. In one or more embodiments, the composition may include an adjuvant, an excipient, an immunomodulator, a checkpoint protein, an antagonist of PD-1 (e.g., an anti-PD-1 antibody), and/or an antagonist of PD-L1 (e.g., an anti-PD-L1 antibody).

In one or more embodiments, the pharmaceutically acceptable composition that is developed and/or manufactured based on the diverse group of candidate peptides (e.g., diverse group of candidate peptides 110 in FIG. 1) identified using the machine learning model(s) (e.g., model 124 in FIG. 1., attention-based machine learning model 201 in FIG. 2) described herein may be a T-cell therapy. For example, the pharmaceutically acceptable composition may include one or more engineered T cells. For example, the pharmaceutically acceptable composition may include a population of engineered T cells.

### V. Methods of Treatment Comprising Immunogenic Vaccines or T Cells

The embodiments described herein provide methods for use in developing a therapeutic for a medical condition (e.g., a disease such as, but not limited to, cancer) and/or treating an individual having the medical condition.

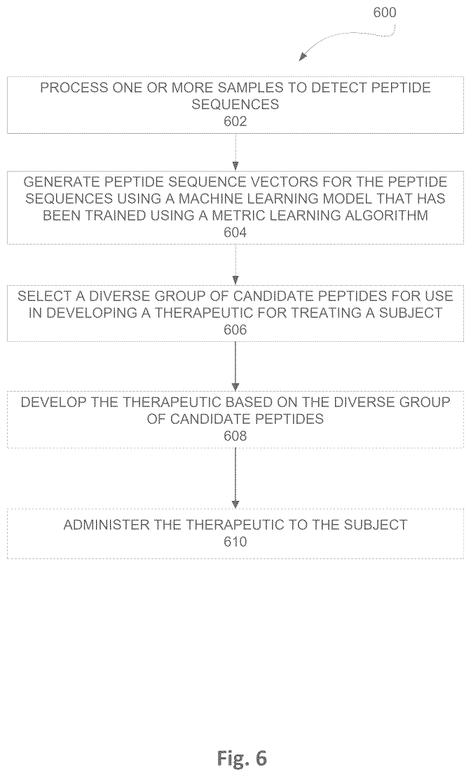

FIG. 6 is a flowchart of a process 600 for treating a subject in accordance with one or more embodiments. Process 600 may be used to treat a subject via a therapeutic developed and/or manufactured based on a diverse group of candidate peptides identified via a machine learning model (e.g., model 124 in FIG. 1, attention-based machine learning model 201 in FIG. 2).

Step 602 includes processing one or more samples to detect peptide sequences. The subject may have a medical condition such as, but not limited to, cancer. The one or more samples may be one or more disease samples.

Step 604 includes generating peptide sequence vectors for the peptide sequences using a machine learning model that has been trained using a metric learning algorithm. The machine learning model may be, for example, model 124 in FIG. 1 or attention-based machine learning model 201 in FIG. 2. The metric learning algorithm may be, for example, metric learning algorithm 126 in FIG. 1. The machine learning model may be trained using, for example, process 400 in FIG. 4 or process 500 in FIG. 5.

Step 606 includes selecting a diverse group of candidate peptides for use in developing a therapeutic for treating a subject. The therapeutic may be, for example, a peptide therapeutic (e.g., peptide therapeutic 108 in FIG. 1) that includes or is based on treatment peptides (e.g., treatment peptides 144 in FIG. 1) that have been selected from the diverse group of candidate peptides (diverse group of candidate peptides 110 in FIG. 1). The peptide therapeutic may include the selected treatment peptides, precursors of the selected treatment peptides, or one or more nucleic acids encoding the treatment peptides or precursors thereof. An individual may be treated by administering an effective amount of a composition (such as a composition described above in Section IV) that includes the selected treatment peptides, precursors of the selected treatment peptides, or one or more nucleic acids encoding the treatment peptides or precursors thereof. The therapeutic may be, for example, a peptide vaccine (e.g., a tumor vaccine) for treating a disease (e.g., cancer).

Process 600 may optionally include step 608 and may optionally include step 610. Step 608 includes developing the therapeutic based on the diverse group of candidate peptides. Step 610 includes administering the therapeutic to the subject.

The individual that is treated may be the same individual from whom the one or more samples (e.g., disease samples) in step 602 was collected. In some instances, the therapeutic is administered to a different individual as compared to the individual from whom the disease sample(s) was collected. The different individual may, for example, be related to the individual from whom the disease sample was collected, have a genetic risk of developing a particular type of cancer, and/or have MHC molecules that have one, more or all alleles corresponding to a sequence that are the same (or similar) to one or more MHC alleles of the subject from who the disease sample was collected.

The therapeutic may be used to treat cancer, which may take any of a number of different forms: carcinoma, lymphoma, blastema, sarcoma, leukemia, squamous cell cancer, lung cancer (including small cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, bladder cancer, hepatoma, breast cancer, colon cancer, melanoma, endometrial or uterine carcinoma, salivary gland carcinoma, kidney or renal cancer, liver cancer, prostate cancer, vulval cancer, thyroid cancer, hepatic carcinoma, head and neck cancer, colorectal cancer, rectal cancer, soft-tissue sarcoma, Kaposi's sarcoma, B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's macroglobulinemia), chronic lymphocytic leukemia (CLL), acute lymphoblastic leukemia (ALL), myeloma, Hairy cell leukemia, chronic myeloblasts leukemia, and post-transplant lymphoproliferative disorder (PTLD), as well as abnormal vascular proliferation associated with phakomatoses, edema (such as that associated with brain tumors), and Meigs' syndrome.

The therapeutic developed in step 608 may be, for example, a vaccine (e.g., a tumor vaccine). The vaccine may include a plurality of peptides; a plurality of precursors for the plurality of peptides; or a set of nucleic acids encoding the plurality of peptides or the plurality of precursors. The plurality of peptides may be selected from among the diverse group of candidate peptides identified in step 606. The plurality of peptides includes at least two peptides that have dissimilar binding motifs.

The vaccine may include DNA that includes the set of nucleic acids (i.e., one or more nucleic acids), RNA that includes the set of nucleic acids, or mRNA that includes the set of nucleic acids. The set of nucleic acids may be identified based on amino acids within the plurality of peptides. The set of nucleic acids may encode the plurality of peptides. In some embodiments, for each peptide of the plurality of peptides, the tumor vaccine includes at least one of: a nucleotide sequence encoding each peptide, an amino acid sequence corresponding to each peptide, RNA corresponding to each peptide, DNA corresponding to each peptide, a cell corresponding to each peptide, a plasmid corresponding to each peptide, or a vector corresponding to each peptide. The vaccine may include at least one of an excipient or an adjuvant. The vaccine may include an RNA molecule. In the 5′→3′ direction, the RNA molecule may include a 5′ cap; a 5′ untranslated region (UTR); a polynucleotide sequence encoding a secretory signal peptide; a polynucleotide sequence encoding the plurality of peptides; a polynucleotide sequence encoding at least a portion of a transmembrane and cytoplasmic domain of a major histocompatibility complex (MHC) molecule; a 3′ UTR that includes a 3′ untranslated region of an Amino-Terminal Enhancer of Split (AES) mRNA or a fragment thereof and non-coding RNA of a mitochondrially encoded 12S RNA or a fragment thereof; and a poly(A) sequence.

In one or more embodiments, the therapeutic that is developed in step 608 is a T cell therapy. The T cell therapy may include a single engineered T cell or multiple (e.g., a population of) engineered T cells. In these embodiments, developing a T cell therapy in step 608 may include, but is not limited to, providing a population of T cells. The cells may be autologous or allogenic to the subject. Step 608 may further include engineering at least a subset of the population of T cells to express an exogenous T cell receptor (TCR) and to knock out an endogenous TCR-beta, thereby forming a population of engineered T cells. The exogenous TCR may bind to an antigen expressed by the cancer and selected based on the diverse group of peptides. The antigen may be, for example, a neoantigen or a TAA (tumor-associated antigen). The presence of the antigen may have been determined by, for example, sequencing at least a portion of the genome and/or transcriptome of cancer. The TCR may bind to the antigen, which is presented on an MHC Class I (MHCI) molecule, which comprises an MHCI allele expressed by the subject.

Step 608 may further include expanding the population of engineered T cells. The expanded population of engineered T cells may include between 1×10⁵ and 1×10¹¹ engineered T cells. In these embodiments, step 610 may then include administering the expanded population of engineered T cells to the subject.

Embodiments disclosed herein can including identifying part or all of and/or implementing part or all of an individualized-medicine strategy. For example, multiple mutant peptides that are dissimilar (e.g., have binding motif diversity) may be selected based on the processing of peptide sequence detected in a sample from an individual. The processing may be performed using a machine learning model such as model 124 in FIG. 1 or attention-based machine learning model 201 in FIG. 2. The mutant peptides and/or precursors thereof may then be administered to the same individual.

In some embodiments, a method of treating a disease such as cancer may include selecting treatment peptides, identifying precursors for the treatment peptides and/or one or more nucleic acid sequences encoding the treatment peptides or precursors thereof, and/or synthesizing the treatment peptides, the precursors of the treatment peptides, or the one or more nucleic acids encoding the treatment peptides or peptide precursors. The synthesized products may then be administered.

### VI. Examples

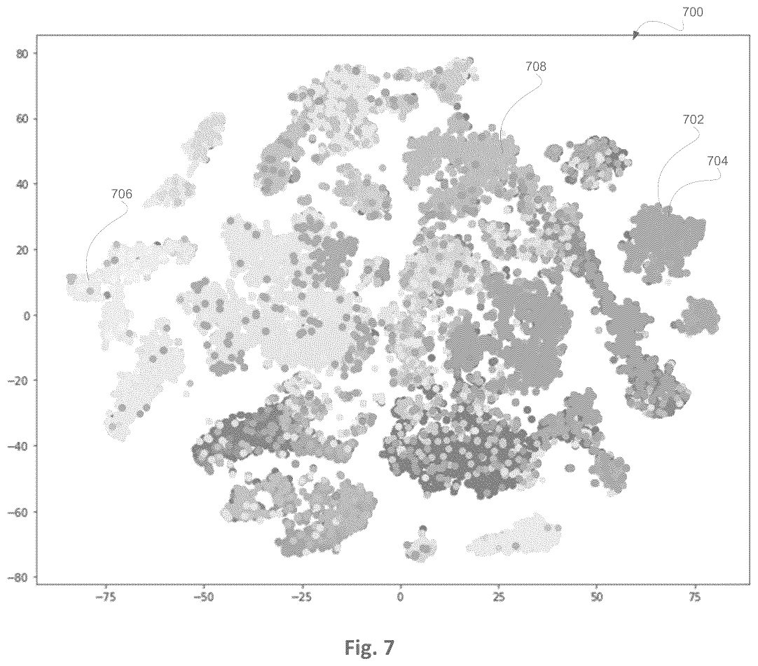

FIG. 7 is an illustration of a plot of peptide sequence vectors in a reduced dimensional space in accordance with one or more embodiments. Plot 700 may be one example of an implementation for a visual representation that is included in output 140 in FIG. 1. Plot 700 shows the similarity relationship between various peptide sequence vectors and thereby, the corresponding peptide sequences represented by these peptide sequence vectors. In one or more embodiments, the peptide sequence vectors are embedded within an n-dimensional space and are visualized in plot 700 in a k-dimensional space. The k-dimensional space is a two-dimensional space, having fewer dimensions than the n-dimensional space. This reduced dimensional space makes it easier to interpret the similarity relationships between the various peptide sequences.

Peptide sequence vectors that are closer together in plot 700 may be more similar and/or be presented by a same or similar MHC allele. More particularly, peptide sequence vectors that are closer together share a same or similar binding motif(s). Peptide sequence vectors that are further apart in plot 700 may be more dissimilar and/or may be presented by different MHC alleles. More particularly, peptide sequence vectors that are further apart have dissimilar binding motifs. The binding motifs may be dissimilar in that the amino acids included in the binding motifs are different, the sequences of the amino acids are different, the spacing (or intervals) between the amino acids is different, or a combination thereof. For example, the binding motifs may be dissimilar by differing by more than a selected number of amino acids (e.g., two, three, four, five, or more amino acids).

In FIG. 7, peptide sequence vector 702 and peptide sequence vector 704 have a same or similar binding motifs. Peptide sequence vector 702 and peptide sequence vector 706 may have dissimilar binding motifs. Peptide sequence vector 708 may have a binding motif that is more similar to the binding motif of peptide sequence vector 702 than the binding motif of peptide sequence vector 706.

In some cases, a peptide sequence for a given peptide may contain multiple binding motifs for binding to a group of MHC alleles. The peptide sequence vector generated for that peptide sequence captures this information. For example, this peptide sequence vector may be located in the n-dimensional space close to peptide sequence vectors for other peptides that also bind to one or more of this group of MHC alleles. Accordingly, these peptide sequence vectors may appear closer together or, in some cases, overlap in the k-dimensional space of plot 700.

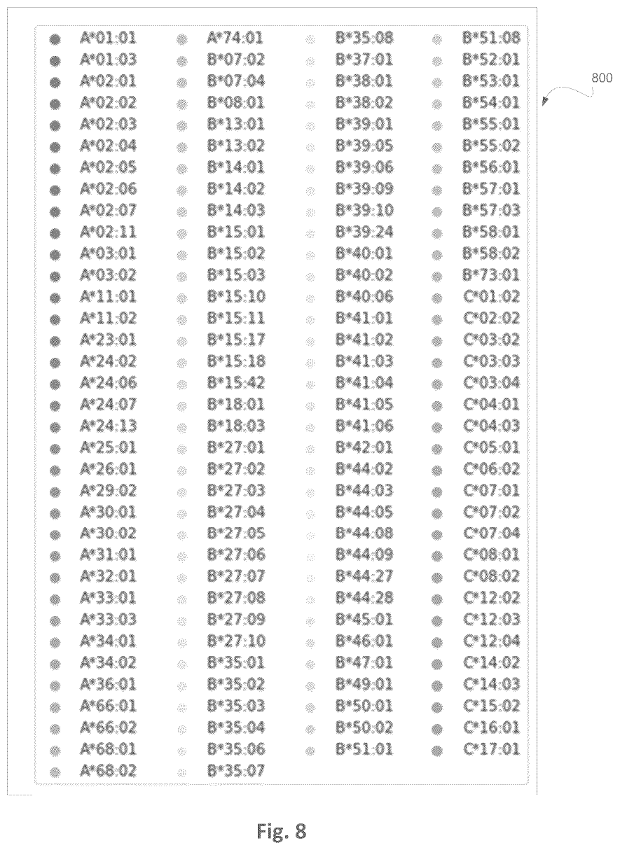

FIG. 8 is a listing of MHC alleles in accordance with one or more embodiments. The listing 800 in FIG. 8 includes the various MHC alleles that present the peptide sequences represented by the peptide sequence vectors included in plot 700.

### VII. Computer Implemented System

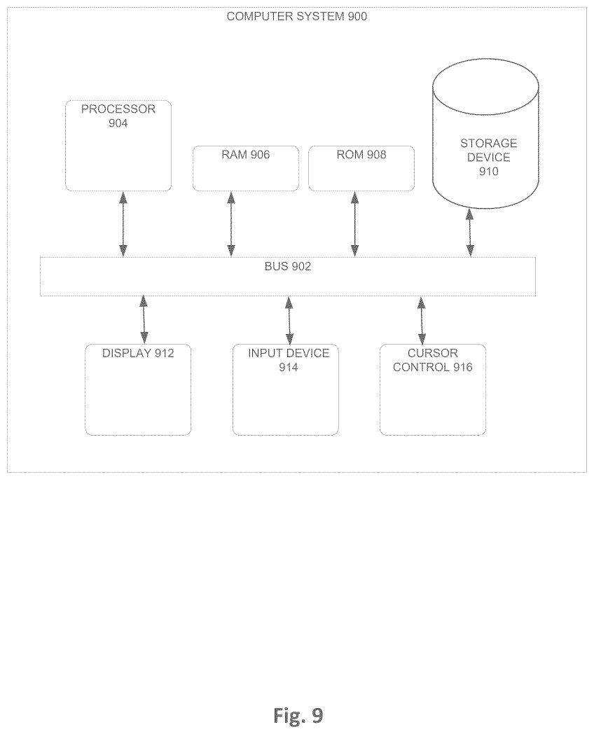

FIG. 9 is a block diagram of a computer system in accordance with various embodiments. Computer system 900 may be an example of one implementation for computing platform 102 described above in FIG. 1.

In one or more examples, computer system 900 can include a bus 902 or other communication mechanism for communicating information, and a processor 904 coupled with bus 902 for processing information. In various embodiments, computer system 900 can also include a memory, which can be a random-access memory (RAM) 906 or other dynamic storage device, coupled to bus 902 for determining instructions to be executed by processor 904. Memory also can be used for storing temporary variables or other intermediate information during execution of instructions to be executed by processor 904. In various embodiments, computer system 900 can further include a read-only memory (ROM) 908 or other static storage device coupled to bus 902 for storing static information and instructions for processor 904. A storage device 910, such as a magnetic disk or optical disk, can be provided and coupled to bus 902 for storing information and instructions.

In various embodiments, computer system 900 can be coupled via bus 902 to a display 912, such as a cathode ray tube (CRT) or liquid crystal display (LCD), for displaying information to a computer user. An input device 914, including alphanumeric and other keys, can be coupled to bus 902 for communicating information and command selections to processor 904. Another type of user input device is a cursor control 916, such as a mouse, a joystick, a trackball, a gesture input device, a gaze-based input device, or cursor direction keys for communicating direction information and command selections to processor 904 and for controlling cursor movement on display 912. This input device 914 typically has two degrees of freedom in two axes, a first axis (e.g., x) and a second axis (e.g., y), that allows the device to specify positions in a plane. However, it should be understood that input devices 914 allowing for three-dimensional (e.g., x, y, and z) cursor movement are also contemplated herein.

Consistent with certain implementations of the present teachings, results can be provided by computer system 900 in response to processor 904 executing one or more sequences of one or more instructions contained in RAM 906. Such instructions can be read into RAM 906 from another computer-readable medium or computer-readable storage medium, such as storage device 910. Execution of the sequences of instructions contained in RAM 906 can cause processor 904 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 904 for execution. Such a medium can take many forms, including but not limited to, non-volatile media, volatile media, and transmission media. Examples of non-volatile media can include, but are not limited to, optical, solid state, magnetic disks, such as storage device 910. Examples of volatile media can include, but are not limited to, dynamic memory, such as RAM 906. Examples of transmission media can include, but are not limited to, coaxial cables, copper wire, and fiber optics, including the wires that comprise bus 902.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 904 of computer system 900 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 900 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 900, whereby processor 904 would execute the analyses and determinations provided by these engines, subject to instructions provided by any one o, or combination of the memory components RAM 906, ROM, 908, or storage device 910 and user input provided via input device 914.

### VIII. Examples of Descriptions of Terms

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology and toxicology are described herein are those well-known and commonly used in the art.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one.

As used herein, the term “plurality” can be 2, 3, 4, 5, 6, 7, 8, 9, 10, or more.

As used herein, the term “set of”' means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

Where reference is made to a list of elements (e.g., elements a, b, c), such reference is intended to include any one of the listed elements by itself, any combination of less than all of the listed elements, and/or a combination of all of the listed elements.

As used herein, a “model” includes at least one of an algorithm, a formula, a mathematical technique, a machine algorithm, a probability distribution or model, or another type of mathematical or statistical representation.

As used herein, a “subject” may refer to or encompass one or more cells, tissue, or an organism. The subject may be a human or non-human, whether in vivo, ex vivo, or in vitro, male or female. A subject can be a mammal, such as a human. A subject may refer to a mammal being assessed for treatment and/or being treated, a mammal participating in a clinical trial, a mammal undergoing anti-cancer therapies, or any other mammal of interest. In various embodiments, the terms “subject,” “individual,” and “patient” are used interchangeably herein. A subject can be a healthy or asymptomatic individual, an individual that has or is suspected of having a disease (e.g., cancer) or a pre-disposition to the disease, an individual that is in need of therapy or suspected of needing therapy, or a combination thereof. A subject may be, for example, without limitation, an individual having cancer or an individual having an autoimmune disease. A subject may be human. In other cases, a subject may be some other type of mammal. For example, a subject may be a mammal used in forming laboratory models for human disease. Such mammals include, but are not limited to, mice, rats, primates (e.g., cynomolgus monkey), etc.

As used herein, a “sample” can refer to “biological sample” of a subject. A sample can include tissue (e.g., a biopsy), single cell, multiple cells, fragments of cells, or an aliquot of body fluid. The sample may be obtained from a subject by means including, for example, without limitation, venipuncture, excretion, ejaculation, massage, biopsy, needle aspirate, lavage sample, scraping, surgical incision, intervention, another type of sample collection means, or a combination thereof.

As used herein, a “nucleotide,” comprises a nucleoside and a phosphate group. A “nucleoside,” as used herein, comprises a nucleobase and a five-carbon sugar (e.g., ribose, deoxyribose, or analogs thereof). When the nucleobase is bonded to ribose, the nucleoside may be referred to as a ribonucleoside. When the nucleobase is bonded to deoxyribose, the nucleoside may be referred to as a deoxyribonucleoside. A “nucleobase,” which may be also referred to as a “nitrogenous base,” can take the form of one of five types: adenine (A), guanine (G), thymine (T), uracil (U), and cytosine (C).

As used herein, a “polynucleotide,” “nucleic acid,” or “oligonucleotide” refers to a linear polymer of nucleotides (or nucleosides joined by internucleosidic linkages). Generally, a polynucleotide comprises at least three nucleotides. Generally, an oligonucleotide is comprised of nucleotides that range in number from a few nucleotides (or monomeric units) to several hundreds of nucleotides (monomeric units). Whenever a polynucleotide such as an oligonucleotide is represented by a sequence of letters, such as “ATGCCTG,” it will be understood that the nucleotides are in 5′→3′ order or direction from left to right and that “A” denotes adenine, “C” cytosine, “G” denotes guanine, and “T” denotes thymine, unless otherwise noted. The letters A, C, G, and T may be used to refer to the nucleobases themselves, as described above, the nucleosides that include those nucleobases, or the nucleotides that include those bases, as is standard in the art.

Deoxyribonucleic acid (DNA) is a chain of nucleotides consisting of 4 types of nucleotides: adenine (A), thymine (T), cytosine (C), and guanine (G). Ribonucleic acid (RNA) is comprised of 4 types of nucleotides: A, C, G, and uracil (U). Certain pairs of nucleotides specifically bind to one another in a complementary fashion, which may be referred to as complementary base pairing. For example, C pairs with G and A pairs with T. In the case of RNA, however, A pairs with U. When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “genomic sequence,” “genetic sequence,” “fragment sequence,” or “nucleic acid sequencing read” denotes any information or data that is indicative of the order of the nucleotide bases (e.g., A, C, G, T/U) in a molecule (e.g., whole genome, whole transcriptome, exome, oligonucleotide, polynucleotide, fragment, etc.) of DNA or RNA. It should be understood that the present disclosure contemplates that this sequence information may be obtained using any of the available varieties of techniques, platforms, or technologies, including, but not limited to: capillary electrophoresis, microarrays, ligation-based systems, polymerase-based systems, hybridization-based systems, direct or indirect nucleotide identification systems, pyrosequencing, ion-or pH-based detection systems, electronic-based systems, etc., or a combination thereof.

A term “genome,” as used herein, refers to the genetic material of a cell or organism, including animals, such as mammals (e.g., humans), and comprises nucleic acids, such as DNA. A genome is stored on one or more chromosomes comprised of DNA sequences. In humans, DNA includes, for example, genes, noncoding DNA, and mitochondrial DNA. The human genome typically contains 23 pairs of chromosomes: 22 pairs of autosomal chromosomes (autosomes) plus the sex-determining X and Y chromosomes. The 23 pairs of chromosomes include one copy from each parent. The DNA that makes up the chromosomes is referred to as chromosomal DNA and is present in the nucleus of human cells (nuclear DNA).

As used herein, a “gene” is a discrete portion of heritable, genomic sequence which affect a subject's traits by being expressed as a functional product or by regulation of gene expression. The total complement of genes in a subject or cell is known as the subject's or cell's genome. A region of a chromosome at which a particular gene is located is called its locus. Each locus contains one allele of a gene. Thus, a pair of chromosomes together has two loci that each contain an allele of the gene to form an allele pair. The two alleles may be the same of may be different (e.g., have slightly varying gene sequences).

As used herein, an “allele” is a variant of a gene. One allele of a gene may differ from another allele of the same gene in various ways. For example, two alleles for a same gene may differ by, for example, a protein (e.g., differences within the amino acid sequence of the encoded protein), other (silent or synonymous) variances in the exon regions that do not affect the amino acid sequence, variances in the intron regions, or some combination of these variances.

As used herein, a “peptide sequence” may refer to an ordered sequence that identifies amino acids for at least a portion of a peptide via, for example, amino acid identifiers, codon identifiers, or nucleotide identifiers. In some cases, the peptide sequence includes a variant-coding sequence that includes a variant that is not observed in a corresponding reference sequence.

When the peptide includes a mutant peptide, the variant-coding sequence, identifies amino acids of the mutation or variant. However, when the peptide does not include a mutation or variant, the variant-coding sequence does not identify amino acids of a mutation or variant (and in that instance is the same as the reference sequence). A variant-coding sequence can be determined by collecting a disease and/or tumor sample (e.g., that includes tumor cells) and performing a sequencing analysis to identify one or more sequences corresponding to disease and/or tumor cells in the sample. In some instances, a sequencing analysis outputs an amino-acid sequence. In some instances, a sequencing analysis outputs a nucleic acid sequence, which may be subsequently processed to transform codons into amino-acid identifiers and thus to produce an amino-acid sequence. A variant-coding sequence can include a sequence of a neoantigen. A variant-coding sequence may, but need not, include one or more termini (e.g., the C-terminus and/or the N-terminus) of the peptide. A variant-coding sequence may include an epitope of the peptide. A variant-coding sequence can identify amino acids within a peptide having one or more variants (e.g., one or more amino-acid distinctions) relative to a corresponding reference sequence. In some instances, a variant-coding sequence includes an ordered set of amino acids. In some instances, a variant-coding sequence identifies a reference peptide (e.g., by identifying a genetic reference sequence, such as by gene, start position and/or end position; or by gene, start position and/or length) and one or more point mutations relative to the reference peptide.

As used herein, a “reference sequence” may refer to a sequence that identifies amino acids within at least part of a non-mutant peptide or wild-type peptide (e.g., wild-type, parental sequence). The non-mutant or wild-type peptide may include no variants or fewer variants than are included in a mutant peptide. The reference sequence may include an amino-acid sequence encoded by a genetic sequence within a same gene relative to a gene that includes a corresponding variant-coding sequence. The reference sequence may include an amino-acid sequence encoded by a genetic sequence spanning a same start and stop within a gene relative to intra-gene positions associated with a genetic sequence associated with a corresponding variant-coding sequence. The reference sequence may be identified by collecting a non-disease and/or non-tumor sample from one or more subjects (who may, but need not, include a subject from which a disease sample was collected to determine a variant-coding sequence) and performing a sequencing analysis using the sample.

As used herein, “MHC” refers to the major histocompatibility complex, which is a system, complex, or group of cell-surface proteins responsible for the regulation of the immune system. The human MHC is also called the human leukocyte antigen (HLA) complex. An HLA system or complex is encoded by the MHC gene complex in humans. MHC molecules that present antigens on cells are categorized as belonging to one of three classes of MHC molecules, MHC class I, MHC class II, and MHC class III. Certain HLA genes including, for example, HLA-A, HLA-B, HLA-C, correspond to MHC class I. Certain HLA genes including, for example, HLA-DP, HLA-DM, HLA-DO, HLA-DQ, and HLADR, correspond to MHC class II. HLA genes that are known include, for example, HLA-A, HLA-B, HLA-C, HLA-E, HLA-F, HLA-G, HLA-H, HLA-J, HLA-K, HLA-L, HLA-N, HLA-P, HLA-S, HLA-T, HLA-U, HLA-V, HLA-W, HLA-X, HLA-Y, HLA-Z, HLA-DRA, HLA-DRB, HLA-DQ, HLA-DOA, HLA-DOB, HLA-DMA, HLA-DMB, HLA-DPA, HLA-DPB, and HFE. Other genes that are found in the HLA region include, for example, TAP1, TAP2, PSMB9, PSMB8, MICA, MICB, MICC, MICD, and MICE.

As used herein, “immunotherapy” refers to a treatment or class of treatments that uses one or more parts of a subject's immune system to fight a disease such as, for example, without limitation, cancer. Immunotherapy can use substances made by the body or synthesized outside of the body to improve how the immune system works to find and destroy cancer cells.

As used herein, a “neoantigen” is a tumor-specific antigen derived from somatic mutations in tumors and presented by a subject's cancer cells and antigen presenting cells. Neoantigen therapies, such as, but not limited to, neoantigen vaccines, are a relatively new approach for providing individualized cancer treatment. Neoantigen vaccines can prime a subject's T cells to recognize and attack cancer cells expressing one or more particular tumor neoantigens. This approach generates a tumor-specific immune response that spares healthy cells while targeting tumor cells. The individualized vaccine may be engineered or selected based on a subject-specific tumor profile. The tumor profile can be defined by determining DNA and/or RNA sequences from a subject's tumor cell and using the sequences to identify neoantigens that are present in tumor cells but absent in normal cells.

As used herein, the terms “peptide,” “polypeptide,” and “protein” may be used interchangeably to refer to a polymer of amino acid residues. The terms encompass amino acid chains of any length, including full-length proteins with amino acid residues linked by covalent peptide bonds.

As used herein, a “mutant peptide” may refer to a peptide that is not present in the normal tissue (e.g., in the wild type amino acid sequences of normal tissue) of an individual subject. A mutant peptide comprises at least one mutant amino acid and may be present in a diseased tissue (e.g., collected from a particular subject) but not in a normal tissue (e.g., collected from the particular subject, collected from a different subject, and/or as identified in a database as corresponding to normal tissue). A mutant peptide may include an epitope. An epitope is the portion of a mutant peptide to which an MHC molecule or a T cell receptor (TCR) binds. Thus, this binding between the epitope of the mutant peptide and the MHC molecule or TCR can induce an immune response (as a result of the mutant peptide not being associated with a subject's “self”). A mutant peptide can include or can be a neoantigen. A mutant peptide can arise from, for example: a non-synonymous mutation leading to different amino acids in the protein (e.g., point mutation); a read-through mutation in which a stop codon is modified or deleted, leading to translation of a longer protein with a novel tumor-specific sequence at the C-terminus; a splice site mutation that leads to a unique tumor-specific protein sequence; a chromosomal rearrangement that gives rise to a chimeric protein with a tumor-specific sequence at a junction of two proteins (i.e., gene fusion) and/or a frameshift insertion or deletion that leads to a new open reading frame with a tumor-specific protein sequence. A mutant peptide can include a polypeptide (as characterized by a polypeptide sequence) and/or may be encoded by a nucleotide sequence.

As used herein, an “epitope” of a peptide may refer to a region of the peptide between the C-flank and N-flank and can be recognized by a TCR. The epitope of the peptide is a part of the peptide that is recognized by TCR on a T cell and MHC I on an antigen presenting cell. For example, the epitope can be a peptide to which a TCR binds, for example, a peptide to which the TCR binds when the peptide is bound to MHC I on an antigen presenting cell.

As used herein, a “representation” of a sequence can include a set of values that represent or identify amino acids in the sequence and/or a set of values that represent or identify nucleic acids that encode the sequence. For example, each amino acid may be represented by a binary string and/or vector of values that is distinct from each other binary string and/or vector representing each other amino acid. The representation may be generated using, for example, one-hot encoding or using a BLOSUM matrix. For example, a multi-dimensional (e.g., 20- or 21-dimensional) array be initialized (e.g., randomly or pseudorandomly initialized). The initialized array may include, for each amino acid, a unique vector corresponding to that amino acid. The values may be fixed such that use of such a unique vector can be assumed to represent the corresponding amino acid. There may be multiple possible nucleic acid representations of a given sequence, given that any of multiple codons can encode a single amino acid.

As used herein, “presentation” of a peptide refers to at least part of the peptide being presented on a surface of a cell by virtue of being bound to an MHC molecule in a particular manner. The presented peptide can then be accessible to other cells, such as nearby T cells.

As used herein, “immunogenicity” may refer to the ability to elicit an immune response (e.g., via T cells and/or B cells). A peptide that is “immunogenic” may be one that is capable of eliciting an immune response.

### IX. Recitation of Example Embodiments

Embodiment 1. A method for developing a therapeutic, the method comprising: receiving peptide sequence data identifying a plurality of peptide sequences that correspond to a plurality of peptides; generating, via a trained machine learning model, a plurality of peptide sequence vectors in an n-dimensional space for respective ones of the plurality of peptide sequences and thereby, for respective ones of the plurality of peptides, wherein the machine learning model has been trained using a metric learning algorithm, training peptide sequence data, and training allele presentation data; wherein the training peptide sequence data identifies a training peptide sequence that corresponds to each training peptide of a plurality of training peptides; wherein the training allele presentation data identifies, for each training peptide sequence of the plurality of training peptides in the training peptide sequence data, one or more major histocompatibility complex (MHC) alleles expected to present a training peptide that corresponds to the respective training peptide sequence; and wherein the metric learning algorithm has been used to train the machine learning model to output the plurality of peptide sequence vectors in the n-dimensional space such that a first distance between a first pair of the peptide sequence vectors in the plurality of peptide sequence vectors generated within the n-dimensional space for a first respective pair of the peptides that are presented by a same MHC allele is less than a second distance between a second pair of the peptide sequence vectors in the plurality of peptide sequence vectors generated within the n-dimensional space for a second pair of the peptides that are presented by different MHC alleles; and generating an output using the plurality of peptide sequence vectors, the output providing an indication of similarity between the peptide sequences for use in selecting a group of candidate peptides from the plurality of peptides for development of the therapeutic.

Embodiment 2. The method of embodiment 1, further comprising: selecting the group of candidate peptides from the plurality of peptides for the development of the therapeutic based on the output such that at least two candidate peptides of the group of candidate peptides have dissimilar binding motifs.

Embodiment 3. The method of embodiment 1 or 2, further comprising: selecting the group of candidate peptides from the plurality of peptides for the development of the therapeutic based on the output such that a bias towards any single binding motif is reduced.

Embodiment 4. The method of embodiment 2 or 3, wherein selecting the group of candidate peptides from the plurality of peptides comprises: identifying a plurality of clusters of the plurality of peptide sequence vectors using the output; and selecting at least one peptide sequence vector from each of the clusters for use in the development of the therapeutic.

Embodiment 5. The method of embodiment 4, wherein selecting the at least one peptide sequence vector for a selected cluster of the plurality of clusters comprises: selecting one peptide sequence vector from the selected cluster that is nearest a center of the selected cluster, wherein the center is selected from one of a centroid, a mean center, a median center, and a density-based center.

Embodiment 6. The method of embodiment 4 or 5, wherein selecting the at least one peptide sequence vector for a selected cluster of the clusters comprises: selecting at least two peptide sequence vectors from the selected cluster, wherein either: each of the at least two peptide sequence vectors is halfway between a center of the cluster and an edge of the cluster; or each of the at least two peptide sequence vectors is located along an edge of the cluster.

Embodiment 7. The method of any one of embodiments 1-6, wherein the therapeutic includes at least two candidate peptides of the group of candidate peptides, the at least two candidate peptides having dissimilar binding motifs.

Embodiment 8. The method of any one of embodiments 1-7, wherein training the machine learning model comprises: training the machine learning model using the metric learning algorithm, the training peptide sequence data, and the training allele presentation data.

Embodiment 9. The method of embodiment 8, wherein training the machine learning model comprises: computing distance metrics for pairs of training peptide sequence vectors in a batch of training peptide sequence vectors; forming an evaluation bundle from the batch of training peptide sequence vectors based on the distance metrics and a mining strategy; evaluating loss for the evaluation bundle using the metric learning algorithm; and updating parameters of the machine learning model based on the loss.

Embodiment 10. The method of embodiment 9, wherein training the machine learning model comprises: repeating the computing, forming, and evaluating steps for a plurality of batches of training peptide sequence vectors formed from training peptide sequence data.

Embodiment 11. The method of embodiment 10 wherein the updating step is performed after the batches have been processed.

Embodiment 12. The method of embodiment 10, wherein the updating step is performed for each of the batches.

Embodiment 13. The method of any one of embodiments 9-12, wherein the mining strategy used for a first portion of the batches is semi-hard negative mining and the mining strategy used for a second portion of the batches processed after the first portion is hard negative mining.

Embodiment 14. The method of any one of embodiments 1-13, wherein the metric learning algorithm comprises at least one of a contrastive loss function, a triplet loss function, a quadruplet loss function, a circle loss function, a multi-class n-pair loss function, a lifted structure loss function, an angular loss function, a divergence loss function, or a constellation loss function.

Embodiment 15. The method of any one of embodiments 1-14, further comprising: training the machine learning model using the metric learning algorithm and a sampling strategy that prioritizes pairs of more similar peptide sequence vectors that have different presenting major histocompatibility complex (MHC) alleles as compared to more dissimilar peptide sequence vectors that have different presenting MHC alleles.

Embodiment 16. The method of any one of embodiments 1-15, wherein generating, via the trained machine learning model, the peptide sequence vectors comprises: generating, via the trained machine learning model, an n-dimensional vector for a peptide of the peptides using at least one of an embedding layer, a positional encoder, a transformer encoder, a self-attention layer, an add and normalization layer, a feed forward layer, a fully connected layer, an activation layer, or a dropout layer.

Embodiment 17. The method of any one of embodiments 1-16, wherein generating, via the trained machine learning model, the plurality of peptide sequence vectors comprises: converting a peptide sequence of the plurality of peptide sequences into a peptide representation that represents the peptide sequence; and converting the peptide representation into the peptide sequence vector for the peptide sequence.

Embodiment 18. The method of any one of embodiments 1-17, wherein the trained machine learning model comprises at least one of a convolutional neural network, a recurrent neural network, or a feed forward neural network.

Embodiment 19. The method of any one of embodiments 1-18, wherein the trained machine learning model comprises an attention-based machine learning model.

Embodiment 20. The method of any one of embodiments 1-19 further comprising: generating the training allele presentation data via a presentation model trained to identify the one or more MHC alleles that is expected to present a peptide based on a peptide sequence identified for the peptide.

Embodiment 21. The method of any one of embodiments 1-20, wherein the plurality of peptide sequences are detected via processing of a disease sample that includes tissue.

Embodiment 22. The method of any one of embodiments 1-21, further comprising: generating a treatment recommendation for the subject that identifies a peptide vaccine that includes at least two candidate peptides of the group of candidate peptides.

Embodiment 23. The method of any one of embodiments 1-22, wherein the therapeutic is a peptide vaccine and further comprising: generating a report based on the output, wherein the report identifies the group of candidate peptides.

Embodiment 24. The method of embodiment 23, further comprising: initiating an action based on the report that facilitates manufacture of the peptide vaccine.

Embodiment 25. The method of embodiment 24, wherein initiating the action comprises: generating an alert that triggers a computerized process involved in the manufacture of the peptide vaccine.

Embodiment 26. The method of any one of embodiments 1-25, further comprising: generating a report based on the output in which the report identifies the group of candidate peptides; and sending the report to a computing platform over a set of communications links that includes at least one of a wired communications link or a wireless communications link.

Embodiment 27. The method of any one of embodiments 1-26, wherein the therapeutic is selected from a group consisting of a T cell therapy, a personalized cancer therapy, an antigen-specific immunotherapy, an antigen-dependent immunotherapy, a vaccine, and a natural killer (NK) cell therapy.

Embodiment 28. The method of any one of embodiments 1-27, further comprising: generating a report based on the output, the report identifying the group of candidate peptides; and manufacturing the therapeutic to include a plurality of treatment peptides selected from the group of candidate peptides, a plurality of precursors for the treatment peptides, or at least one nucleic acid encoding the plurality of treatment peptides or the precursors, wherein the plurality of treatment peptides includes at least two dissimilar peptides.

Embodiment 29. The method of any one of embodiments 1-28, further comprising: sequencing a disease sample from a subject; defining the plurality of peptide sequences based on the sequencing of the disease sample from the subject; synthesizing mRNA that codes for at least two candidate peptides included in the group of candidate peptides; complexing the mRNA with lipids to produce an mRNA-lipoplex treatment; and administering the mRNA-lipoplex treatment to the subject.

Embodiment 30. A method for developing a peptide vaccine, the method comprising: training a machine learning model using a metric learning algorithm, training peptide sequence data, and training allele presentation data corresponding to the training peptide sequence data; receiving peptide sequence data identifying a plurality of peptide sequences that correspond to a plurality of peptides; generating, via the machine learning model, a peptide sequence vector for each peptide sequence of the plurality of peptide sequences using the peptide sequence data to form a plurality of peptide sequence vectors; generating an output using the plurality of peptide sequence vectors, the output providing an indication of similarity between peptide sequences of the plurality of peptide sequences; and selecting a group of candidate peptides from the plurality of peptides for development of the peptide vaccine based on the output such that the group of candidate peptides includes at least two dissimilar candidate peptides.

Embodiment 31. A method comprising: receiving training peptide sequence data comprising a plurality of training peptide sequences; generating training allele presentation data for the training peptide sequence data, wherein the training allele presentation data identifies, for a training peptide sequence of the plurality of training peptide sequences, an MHC allele that is predicted to present the training peptide sequence; and training a machine learning model using the training peptide sequence data, the training allele presentation data, and a metric learning algorithm, wherein the machine learning model is trained to generate a peptide sequence vector for a given peptide sequence; and wherein the peptide sequence vector is a vector in an n-dimensional space that provides an indication of a similarity of the given peptide sequence to other peptide sequences.

Embodiment 32. The method of embodiment 31, further comprising: generating, via the trained machine learning model, a plurality of peptide sequence vectors for a plurality of peptide sequences detected in a disease sample; and generating an output using the plurality of peptide sequence vectors, the output providing an indication of similarity between peptide sequences of the plurality of peptide sequences.

Embodiment 33. The method of embodiment 32, further comprising: selecting a group of candidate peptides from the plurality of peptides for development of a peptide vaccine based on the output such that the group of candidate peptides includes at least two candidate peptides with dissimilar binding motifs.

Embodiment 34. The method of embodiment 32 or 33, further comprising: selecting a group of candidate peptides from the plurality of peptides for development of a peptide vaccine based on the output such that a bias towards any single binding motif is reduced in the group of candidate peptides.

Embodiment 35. The method of any one of embodiments 31-34, wherein the training comprises: computing distance metrics for pairs of training peptide sequence vectors in a batch of training peptide sequence vectors; forming an evaluation bundle from the batch of training peptide sequence vectors based on the distance metrics and a mining strategy; evaluating loss for the evaluation bundle using the metric learning algorithm; and updating parameters of the machine learning model based on the loss.

Embodiment 36. The method of any one of embodiments 31-35, wherein the training comprises: training the machine learning model using the metric learning algorithm and a sampling strategy that prioritizes pairs of more similar peptide sequence vectors that have different presenting major histocompatibility complex (MHC) alleles as compared to more dissimilar peptide sequence vectors that have different presenting MHC alleles.

Embodiment 37. The method of any one of embodiments 31-36, wherein the metric learning algorithm comprises at least one of a contrastive loss function, a triplet loss function, a quadruplet loss function, a circle loss function, a multi-class n-pair loss function, a lifted structure loss function, an angular loss function, a divergence loss function, or a constellation loss function.

Embodiment 38. The method of any one of embodiments 31-37, further comprising: generating the training allele presentation data via a presentation model trained to identify the one or more MHC alleles that is expected to present a peptide based on a peptide sequence identified for the peptide.

Embodiment 39. A vaccine comprising: a plurality of peptides; a plurality of precursors for the plurality of peptides; or a set of nucleic acids encoding the plurality of peptides or the plurality of precursors, wherein the plurality of peptides are selected from among the group of candidate peptides selected based on the method of any of embodiments 1-38, and wherein the plurality of peptides includes at least two peptides that have dissimilar binding motifs.

Embodiment 40. The vaccine of embodiment 39, wherein the vaccine includes either DNA that includes the set of nucleic acids or RNA that includes the set of nucleic acids.

Embodiment 41. The vaccine of embodiments 39 or 40, wherein the vaccine includes mRNA that includes the set of nucleic acids.

Embodiment 42. The vaccine of any one of embodiments 39-41, wherein the vaccine is a tumor vaccine.

Embodiment 43. A method of manufacturing a vaccine comprising: producing a vaccine comprising: a plurality of peptides; a plurality of precursors for the plurality of peptides; or a set of nucleic acids encoding the plurality of peptides or the plurality of precursors, wherein the plurality of peptides are selected from among the group of candidate peptides selected based on the method of any of embodiments 1-38, and wherein the plurality of peptides includes at least two peptides that have dissimilar binding motifs.

Embodiment 44. The method of embodiment 43, wherein the vaccine includes DNA that includes the set of nucleic acids, RNA that includes the set of nucleic acids, or mRNA that includes the set of nucleic acids.

Embodiment 45. The method of embodiments 43 or 44, further comprising: identifying, based on amino acids within the plurality of peptides, the set of nucleic acids that the encode the plurality of peptides, wherein the vaccine includes the set of nucleic acids.

Embodiment 46. The method of any one of embodiments 43-45, wherein the vaccine is a tumor vaccine.

Embodiment 47. The method of embodiment 46, wherein, for each peptide of the plurality of peptides, the tumor vaccine comprises at least one of: a nucleotide sequence encoding each peptide, an amino acid sequence corresponding to each peptide, RNA corresponding to each peptide, DNA corresponding to each peptide, mRNA corresponding to each peptide, a cell corresponding to each peptide, a plasmid corresponding to each peptide, or a vector corresponding to each peptide.

Embodiment 48. The method of any one of embodiments 43-47, wherein the vaccine further includes at least one of an excipient or an adjuvant.

Embodiment 49. The method of any one of embodiments 43-48, wherein the vaccine includes an RNA molecule including, in the 5′→3′ direction: a 5′ cap; a 5′ untranslated region (UTR); a polynucleotide sequence encoding a secretory signal peptide; a polynucleotide sequence encoding the plurality of peptides; a polynucleotide sequence encoding at least a portion of a transmembrane and cytoplasmic domain of a major histocompatibility complex (MHC) molecule; a 3′ UTR including: a 3′ untranslated region of an Amino-Terminal Enhancer of Split (AES) mRNA or a fragment thereof; and non-coding RNA of a mitochondrially encoded 12S RNA or a fragment thereof; and a poly(A) sequence.

Embodiment 50. A pharmaceutical composition comprising two or more peptides selected from the group of candidate peptides that is selected based on the method of any of embodiments 1-38.

Embodiment 51. A pharmaceutical composition comprising two or more nucleic acid sequences that encodes two or more respective peptides selected from the group of candidate peptides that is selected based on the method of any of embodiments 1-38.

Embodiment 52. A method of treating a subject comprising administering at least one of two or more peptides, two or more pharmaceutical compositions, or two or more nucleic acid sequences identified based on the output generated by the method of any of embodiments 1-38.

Embodiment 53. An engineered T cell made using the method of any one of embodiments 1-38.

Embodiment 54. A population of engineered T cells made using the method of any one of embodiments 1-38.

Embodiment 55. A method for treating a subject having cancer, the method comprising: providing a population of T cells; engineering at least a subset of the population of T cells to express an exogenous T cell receptor (TCR) and to knock out an endogenous TCR-beta, thereby forming a population of engineered T cells, wherein the exogenous TCR binds to an antigen expressed by the cancer and selected using the method of any one of embodiments 1-38; expanding the population of engineered T cells; and administering the expanded population of engineered T cells to the subject.

Embodiment 56. The method of embodiment 55, wherein the antigen is a neoantigen or a TAA.

Embodiment 57. The method of embodiment 55 or 56, wherein at least a portion of the genome and/or transcriptome of the cancer was sequenced to determine the presence of the antigen.

Embodiment 58. The method of any one of embodiments 55-57, wherein the engineered T cells are made using the method of any one of embodiments 1-38.

Embodiment 59. The method of any one of embodiments 55-58, wherein the TCR binds to the antigen presented on a major histocompatibility complex class I (MHCI) molecule.

Embodiment 60. The method of embodiment 59, wherein the MHCI comprises an MHCI allele expressed by the subject.

Embodiment 61. The method of any one of embodiments 55-60, wherein the expanded population of engineered T cells comprises between 1×10⁵ and 1×10¹¹ engineered T cells.

Embodiment 62. The method of any one of embodiments 55-61, wherein the T cells are autologous to the subject.

Embodiment 63. The method of any one of embodiments 55-61, wherein the T cells are allogenic to the subject.

Embodiment 64. A method of treating cancer comprising administering a T cell, composition, or pharmaceutical composition, of any of embodiments 55-63 to a patient having a cancer.

Embodiment 65. A system comprising: one or more data processors; and a non-transitory computer readable storage medium containing instructions which, when executed on the one or more data processors, cause the one or more data processors to perform the method of any one of embodiments 1-38.

Embodiment 66. A computer-program product tangibly embodied in a non-transitory machine-readable storage medium, including instructions configured to cause one or more data processors to perform the method of any one of embodiments 1-38.

### X. Additional Considerations

The headers and subheaders between sections and subsection of this document are included solely for the purpose of improving readability and do not imply that features cannot be combined across sections and subsections. Accordingly, sections and subsections do not describe separate embodiments.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The description provides preferred example embodiments only, and is not intended to limit the scope, applicability, or configuration of the disclosure. Rather, the description of the preferred example embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

What is claimed is:

1. A method for developing a therapeutic, the method comprising:
receiving peptide sequence data identifying a plurality of peptide sequences that correspond to a plurality of peptides;
generating, via a trained machine learning model, a plurality of peptide sequence vectors in an n-dimensional space for respective ones of the plurality of peptide sequences and thereby, for respective ones of the plurality of peptides,
wherein the machine learning model has been trained using a metric learning algorithm, training peptide sequence data, and training allele presentation data;
wherein the training peptide sequence data identifies a training peptide sequence that corresponds to each training peptide of a plurality of training peptides;
wherein the training allele presentation data identifies, for each training peptide sequence of the plurality of training peptides in the training peptide sequence data, one or more major histocompatibility complex (MHC) alleles expected to present a training peptide that corresponds to the respective training peptide sequence; and
wherein the metric learning algorithm has been used to train the machine learning model to output the plurality of peptide sequence vectors in the n-dimensional space such that a first distance between a first pair of the peptide sequence vectors in the plurality of peptide sequence vectors generated within the n-dimensional space for a first respective pair of the peptides that are presented by a same MHC allele is less than a second distance between a second pair of the peptide sequence vectors in the plurality of peptide sequence vectors generated within the n-dimensional space for a second pair of the peptides that are presented by different MHC alleles; and
generating an output using the plurality of peptide sequence vectors, the output providing an indication of similarity between the peptide sequences for use in selecting a group of candidate peptides from the plurality of peptides for development of the therapeutic.

2. The method of claim 1, further comprising:
selecting the group of candidate peptides from the plurality of peptides for the development of the therapeutic based on the output such that at least two candidate peptides of the group of candidate peptides have dissimilar binding motifs.

3. The method of claim 1, further comprising:
selecting the group of candidate peptides from the plurality of peptides for the development of the therapeutic based on the output such that a bias towards any single binding motif is reduced.

4. The method of claim 2, wherein selecting the group of candidate peptides from the plurality of peptides comprises:
identifying a plurality of clusters of the peptide sequence vectors using the output; and selecting at least one peptide sequence vector from each of the plurality of clusters for use in the development of the therapeutic.

5. The method of claim 1, wherein the therapeutic includes at least two candidate peptides of the group of candidate peptides, the at least two candidate peptides having dissimilar binding motifs.

6. The method of claim 1, wherein training the machine learning model comprises:
training the machine learning model using the metric learning algorithm, the training peptide sequence data, and the training allele presentation data.

7. The method of claim 1, wherein the metric learning algorithm comprises at least one of a contrastive loss function, a triplet loss function, a quadruplet loss function, a circle loss function, a multi-class n-pair loss function, a lifted structure loss function, an angular loss function, a divergence loss function, or a constellation loss function.

8. The method of claim 1, further comprising:
training the machine learning model using the metric learning algorithm and a sampling strategy that prioritizes pairs of more similar peptide sequence vectors that have different presenting major histocompatibility complex (MHC) alleles as compared to more dissimilar peptide sequence vectors that have different presenting MHC alleles.

9. The method of claim 1, wherein generating, via the trained machine learning model, the plurality of peptide sequence vectors comprises:
generating, via the trained machine learning model, an n-dimensional vector for a peptide of the peptides using at least one of an embedding layer, a positional encoder, a transformer encoder, a self-attention layer, an add and normalization layer, a feed forward layer, a fully connected layer, an activation layer, or a dropout layer.

10. The method of claim 1, wherein generating, via the trained machine learning model, the plurality of peptide sequence vectors comprises:
converting a peptide sequence of the plurality of peptide sequences into a peptide representation that represents the peptide sequence; and
converting the peptide representation into the peptide sequence vector for the peptide sequence.

11. The method of claim 1, wherein the trained machine learning model comprises at least one of a convolutional neural network, a recurrent neural network, or a feed forward neural network.

12. The method of claim 1, wherein the trained machine learning model comprises an attention-based machine learning model.

13. The method of claim 1, further comprising:
generating the training allele presentation data via a presentation model trained to identify the one or more MHC alleles that is expected to present a peptide based on a peptide sequence identified for the peptide.

14. The method of claim 1, wherein the plurality of peptide sequences are detected via processing of a disease sample that includes tissue.

15. The method of claim 1, further comprising:
generating a treatment recommendation for the subject that identifies a peptide vaccine that includes at least two candidate peptides of the group of candidate peptides.

16. The method of claim 1, wherein the therapeutic is a peptide vaccine and further comprising:
generating a report based on the output, wherein the report identifies the group of candidate peptides.

17. The method of claim 1, wherein the therapeutic is selected from a group consisting of a T cell therapy, a personalized cancer therapy, an antigen-specific immunotherapy, an antigen-dependent immunotherapy, a vaccine, and a natural killer (NK) cell therapy.

18. The method of claim 1, further comprising:
generating a report based on the output, the report identifying the group of candidate peptides; and
manufacturing the therapeutic to include a plurality of treatment peptides selected from the group of candidate peptides, a plurality of precursors for the plurality of treatment peptides, or at least one nucleic acid encoding the plurality of treatment peptides or the plurality of precursors, wherein the plurality of treatment peptides includes at least two dissimilar peptides.

19. The method of claim 1, further comprising: sequencing a disease sample from a subject;
defining the plurality of peptide sequences based on the sequencing of the disease sample from the subject;
synthesizing mRNA that codes for at least two candidate peptides included in the group of candidate peptides;
complexing the mRNA with lipids to produce an mRNA-lipoplex treatment; and administering the mRNA-lipoplex treatment to the subject.

20. A system comprising:
one or more data processors; and
a non-transitory computer readable storage medium containing instructions which, when executed on the one or more data processors, cause the one or more data processors to perform a method comprising:
receiving peptide sequence data identifying a plurality of peptide sequences that correspond to a plurality of peptides;
generating, via a trained machine learning model, a plurality of peptide sequence vectors in an n-dimensional space for respective ones of the plurality of peptide sequences and thereby, for respective ones of the plurality of peptides,
wherein the machine learning model has been trained using a metric learning algorithm, training peptide sequence data, and training allele presentation data;
wherein the training peptide sequence data identifies a training peptide sequence that corresponds to each training peptide of a plurality of training peptides;
wherein the training allele presentation data identifies, for each training peptide sequence of the plurality of training peptides in the training peptide sequence data, one or more major histocompatibility complex (MHC) alleles expected to present a training peptide that corresponds to the respective training peptide sequence; and
wherein the metric learning algorithm has been used to train the machine learning model to output the plurality of peptide sequence vectors in the n-dimensional space such that a first distance between a first pair of the peptide sequence vectors in the plurality of peptide sequence vectors generated within the n-dimensional space for a first respective pair of the peptides that are presented by a same MHC allele is less than a second distance between a second pair of the peptide sequence vectors in the plurality of peptide sequence vectors generated within the n-dimensional space for a second pair of the peptides that are presented by different MHC alleles; and
generating an output using the plurality of peptide sequence vectors, the output providing an indication of similarity between the peptide sequences for use in selecting a group of candidate peptides from the plurality of peptides for development of the therapeutic.

21. A computer-program product tangibly embodied in a non-transitory machine-readable storage medium, including instructions configured to cause one or more data processors to perform a method comprising:
receiving peptide sequence data identifying a plurality of peptide sequences that correspond to a plurality of peptides;
generating, via a trained machine learning model, a plurality of peptide sequence vectors in an n-dimensional space for respective ones of the plurality of peptide sequences and thereby, for respective ones of the plurality of peptides,
wherein the machine learning model has been trained using a metric learning algorithm, training peptide sequence data, and training allele presentation data;
wherein the training peptide sequence data identifies a training peptide sequence that corresponds to each training peptide of a plurality of training peptides;
wherein the training allele presentation data identifies, for each training peptide sequence of the plurality of training peptides in the training peptide sequence data, one or more major histocompatibility complex (MHC) alleles expected to present a training peptide that corresponds to the respective training peptide sequence; and
wherein the metric learning algorithm has been used to train the machine learning model to output the plurality of peptide sequence vectors in the n-dimensional space such that a first distance between a first pair of the peptide sequence vectors in the plurality of peptide sequence vectors generated within the n-dimensional space for a first respective pair of the peptides that are presented by a same MHC allele is less than a second distance between a second pair of the peptide sequence vectors in the plurality of peptide sequence vectors generated within the n-dimensional space for a second pair of the peptides that are presented by different MHC alleles; and
generating an output using the plurality of peptide sequence vectors, the output providing an indication of similarity between the peptide sequences for use in selecting a group of candidate peptides from the plurality of peptides for development of the therapeutic.
